SEQUENCE LISTING (58 sequences)

SEQ ID NO: 1 - protein, 547 aa, Artificial sequence
  TM4gp29 Lysin A
MSFTRFLQDDPLLTREQVMAELIRVADELNMPDKRGACVIAGMTISQEVGVKDNDPPFERRFWCPANRADPESFNYPHDSESNDGRSVGYFQQQKGPNGELWWGTTASEMNLHSAATQFMTRLKAAGYNASNAQAANDSAQAIQRSGVPQAYKQWWDDINRLYDKVKGSGGGPAPAPKPPQSGPWTGDPVWLADVLRAEGLNVVELPGWLDRGHGDMGRLWGVVCHHTGSDNTPSSEIAFHPSLGLCSQIHLARNGTVTLCGVGIAWHAGVGSYPGLPEDNANAVTIGIEAQNSGTYDGAPHRTNWPDAQYDAYVKCCAAICRRLGVRADHVISHKEWAGRKQGKWDPGAIDMNIFRADVQRRIDAHQPNGEDDFMAALSADEQREVLNLLRVLADRRFVSRSPFRHLGEGPSETVAGFGLNTDGLNHAQYTIELARLGDPTHLALLREVASAEGDSRYPDRQYDAKLAKRVLAEIEGAATAPAKPSTPSAPTEPAPEAPTPPVKAACALSAAGCVVAGSTSGGGCALSTDGTGKCVVTAATDGGAA

SEQ ID NO: 2 - DNA, 1644 bp, Artificial sequence
  TM4gp29 Lysin A
atgagtttcacccggttcctgcaggatgacccgctgctcacccgcgagcaagtgatggccgagctgattcgggtcgccgacgagctgaacatgcccgacaagcgcggcgcctgcgtcattgcgggcatgacgatttcgcaagaggtcggcgtaaaggacaacgacccgccgttcgagcggcggttctggtgcccggccaaccgcgccgaccccgaatcgttcaactacccgcacgactcggaatcgaacgacggccgctcggtcggctacttccagcagcagaaggggcctaacggcgagctgtggtggggcacaacggcatccgagatgaacctgcacagcgccgcgacgcagtttatgacgcggctcaaggcggccggatacaacgcgagcaacgcccaggcggcgaacgactcggcgcaggcgatccagcggtcgggcgtcccgcaggcgtacaagcaatggtgggacgacattaaccgcctgtacgacaaggtgaagggctcgggcggtggcccggcgcccgcgcctaagccgccgcagtcggggccgtggaccggcgacccggtgtggctggccgacgtgctgcgcgccgaggggctgaacgtcgtcgagctgcccggctggctcgaccgcgggcacggcgacatgggccgcttgtggggcgtggtgtgccatcacaccggcagcgataacaccccgtcgagcgagattgcgtttcacccgtcgctcggcctgtgctcgcagattcacctggcgcgcaacggaactgtgacgctgtgcggtgtcggcatcgcctggcatgcgggcgtcggcagctatcccggcctgcccgaggacaacgccaacgcggtcactatcggcatcgaggcccaaaacagcggcacctatgacggcgcaccgcaccggacgaattggcctgacgcgcaatacgacgcctatgtgaagtgctgcgccgcgatctgccgccgcctcggcgtgcgcgccgatcacgtgatcagtcacaaggaatgggccgggcgcaagcaaggcaaatgggatccaggcgccatcgacatgaacatctttcgcgccgacgtacagcggcgcatcgacgcccatcaaccaaacggagaggacgatttcatggccgcactatcagccgacgagcagcgcgaggtgctgaacctgctgcgcgtcctggccgaccggcggttcgtcagccgcagcccgttccgccaccttggcgaggggccgagcgaaactgtcgccgggttcgggctcaacaccgacggcctcaatcacgcgcagtacacgattgagcttgcgcgcctgggcgacccgacgcacctcgccctgctgcgcgaggtcgccagcgccgagggtgactcgcgctatcccgaccggcagtacgacgccaagctcgccaagcgcgtgctcgccgaaatcgagggcgccgcaacggcaccggccaagccgagcacgccgagcgccccgaccgagcccgcccccgaggcgcccacgccgccggtcaaggccgcgtgtgcgctgtctgcggccgggtgcgtggtggctggctcgacctcgggcggtggctgcgccctgtccaccgacggcaccggcaagtgcgttgtgaccgccgcgaccgacggcggggccgcctga

SEQ ID NO: 3 - protein, 514 aa, Artificial sequence
  BXZ2gp11 Lysin A
MTEKVLPYDRSIVTQETGWWCGPAATQVVLNSRGIIVPEATLAAEIEAIENPGRGDDRDGTDYVGLIEQVLDRRVPQARYTSVYLTNDPPTQAQKDRLWEHIVRSINAGYGVVMNWVAPPSNKPRGVKGSVSPRYSGGTTYHYVACMGYDDTPGARAVWIADSGFQPQGYWISFDQCATLIPPKGYAYADAAPAAPAPAPTPVVDAAPILARAAGISEAKAREILPTMRDGLKQADCTTVNRIAMFIAQTGHESDDFRATEEYANGPLDQERWIYKGRTWIQITWREHYARFGKWCFDRGLVTDPDVFVKNPRALADLKWAGIGAAWYWTVERPDINALCDRRDIETVSRRINGTNPNTGRANHIEERIARWNRALAVGDDLLQLIREEEDGFLSALTPAEQRALYNEIMKKGPTRSFMAEDQNQIETLLGFVYNIDGNIWNDAVTRAYLFDVPLAVEYVERVARDGVHPKSWAFQQLDGKGERWLAKFGQEYCKGLIRFKKKLNDLLEPYGEN

SEQ ID NO: 4 - DNA, 1545 bp, Artificial sequence
  Bxz2gp11 Lysin A
atgacggagaaggtacttccctacgaccgcagcatcgtcacgcaggagaccggctggtggtgtggccctgcggccacccaggtcgtgctcaactcgcgaggcatcatcgtcccggaggccacgctcgctgccgagatcgaggccatcgagaaccccggacggggtgacgaccgcgacggcaccgactacgtcggcctgatcgagcaggtgctggatcgccgtgtgccgcaggcgcgctacacgtcggtctacctgacgaacgatccgcccacgcaggctcagaaggaccggctgtgggagcacatcgtccggtcgatcaacgcgggctacggcgtggtcatgaactgggtcgcgcctccgtcgaacaagccacgcggagtgaagggctcggtgagcccgcgctactcgggcggcaccacgtaccactacgtcgcgtgcatgggctacgacgacacccccggtgctcgggcggtctggatcgccgacagcggcttccagccgcagggctactggatctcgttcgaccagtgcgccacgctgatcccgccgaagggctacgcgtacgccgacgccgcaccggctgctcccgcacccgcaccgacccccgtggtcgacgccgcgccgatcctggcgcgtgctgcgggcatctccgaggccaaggcccgcgagatcctgccgacgatgcgtgacgggctgaagcaggccgactgcaccaccgtcaaccggatcgcgatgttcatcgcccagaccggccacgagtccgacgacttccgggccaccgaggagtacgccaacggtcccctggaccaggagcgctggatctacaagggacgcacctggattcagatcacctggcgcgagcactacgcccggttcgggaagtggtgcttcgaccgcggcctggtgaccgaccccgacgtgttcgtcaagaacccgcgtgcgctggccgatctgaagtgggccggcatcggcgcggcctggtactggacggtcgagcgcccggacatcaacgcgctgtgcgaccgccgcgacatcgagacggtctcgcgacggatcaacgggacgaacccgaacaccggacgcgccaaccacatcgaagagcggatcgcccgctggaaccgcgcactcgcggtcggtgacgacctgctgcaacttatccgagaggaggaggacggcttcttgtccgcactcacacccgctgaacagcgcgctctctacaacgagatcatgaagaagggtccgacccggtcgttcatggccgaggaccagaaccagatcgagacgctgctcggcttcgtctacaacatcgacggcaacatctggaacgacgcggtgacccgcgcctacctgttcgacgtgccactggctgttgagtacgtcgagcgcgttgctcgcgacggcgtccacccgaagtcgtgggcgttccagcagctcgacggcaagggcgagcgctggctggccaagttcggccaggagtactgcaagggcctgatccgcttcaagaagaagctgaacgacctgcttgagccgtacggggagaactga

SEQ ID NO: 5 - protein, 493 aa, Artificial sequence
  D29gp10 Lysin A
MTLIVTRDHAQWVHDMCRARAGNRYGYGGAFTLNPRDTTDCSGLVLQTAAWYGGRKDWIGNRYGSTESFRLDHKIVYDLGFRRLPPGGVAALGFTPVMLVGLQHGGGGRYSHTACTLMTMDIPGGPVKVSQRGVDWESRGEVNGVGVFLYDGARAWNDPLFHDFWYLDAKLEDGPTQSVDAAEILARATGLAYNRAVALLPAVRDGLIQADCTNPNRIAMWLAQIGHESDDFKATAEYASGDAYDTRTDLGNTPEVDGDGRLYKGRSWIMITGKDNYRDFSRWAHGRGLVPTPDYFVVHPLELSELRWAGIGAAWYWTVERPDINALSDRRDLETVTRRINGGLTNLDDRRRRYNLALAVGDQLLTLIGDDDELADPTIQRFIREIHGALFNTVVTQSPYGDPQNPDGSEPRSNLWQLHELIKNGDGMGHARYVEESARAGDLRELERVVRAAKGLGRDRSPEFIARARNVLAQIEAANPEYLQAYIARNGAL

SEQ ID NO: 6 - DNA, 1482 bp, Artificial sequence
  D29gp10 Lysin A
atgacgctcatagtcacacgcgaccacgcgcagtgggtccacgacatgtgccgcgctcgcgctggcaacaggtacggctacggcggggcgttcacactcaacccccgagacaccaccgactgctcgggtctggttctgcagacggcagcctggtacggcggtcggaaggactggatcggaaaccggtacggctcgactgagagcttccggctcgaccacaagatcgtctacgacctcgggttcaggcgactccctccgggaggcgttgcggccctgggattcaccccggtcatgctcgtcgggctccagcacggcggcgggggccggtactcgcacaccgcttgcacgctgatgacgatggacatccccggtggcccggtgaaggtctcgcaacgaggcgtcgactgggagtcccgaggagaagtcaacggcgtgggggtgttcctctacgacggcgcacgcgcctggaacgacccgctcttccacgacttctggtacctggacgcgaagcttgaagacggcccgacgcagagtgtcgacgctgccgaaatcctcgctcgcgcaacgggtctcgcgtacaaccgagcggtagcactgctgccggccgtgcgtgacggcctcatccaggccgactgcaccaacccgaatcgcatcgcgatgtggctcgcccagatcggccatgagtcagacgatttcaaggccactgcggagtacgccagcggggacgcctacgacacccgaaccgacctcggcaacaccccggaggtcgacggagacggtcggctctacaagggccggtcctggatcatgatcacgggcaaggacaactaccgggacttctcccggtgggctcacggcaggggcctggtccccacgcccgactacttcgtggttcacccgctggagctgtcggagctgcgctgggcaggcatcggtgccgcctggtactggaccgtcgagcgcccagacatcaacgcactcagcgaccgccgcgacctcgaaacggtcacgcgccggatcaacggcgggctcaccaacctcgatgaccgccgacgccggtacaacctggccctcgctgtgggcgaccaactactgactctgatcggagatgacgacgaattggctgatccaacgattcagcggttcatccgcgagatccacggggcgctgttcaacaccgtcgtgacgcagtccccctacggcgacccgcagaacccggacggctcggagccccggagcaacctctggcagctccatgagctgatcaagaacggcgacggcatggggcacgcccgctacgtcgaggaatcggcgcgagccggtgacctccgcgagctggagcgagttgtccgcgccgccaagggacttggtagggatcgctcccccgagttcatcgcacgcgctcggaacgtgctggcccagatcgaggcagccaaccccgagtacctacaggcgtacatcgccaggaatggagccctatga

SEQ ID NO: 7 - protein, 292 aa, Artificial sequence
  L5gp10 Lysin A
MTFTVTRERAQWVHDMARARDGLPYAYGGAFTNNPRVSTDCSGLVLQTGAWYGGRTDWVGNRYGSTESFRLDHKIVYDLGFKRMPRGGPAALPIKPVMLVGLQHGGGGVYSHTACTLMTMDHPGGPVKMSDRGVDWESHGNRNGVGVELYEGARAWNDPLFHDFWYLDAVLEDEGDDDELADPVLGKMIREIHACLFNQTASTSDLATPGEGAIWQLHQKIHSIDGMLHPIHAERRARAGDLGELHRIVLAAKGLGVKRDEVTKRVYQSILADIERDNPEVLQRYIAERGGL

SEQ ID NO: 8 - DNA, 879 bp, Artificial sequence
  L5gp10 Lysin A
atgaccttcacagtcacccgcgagagagcgcagtgggtccacgacatggcccgcgctcgcgacggtctcccctacgcgtacggcggggcgttcaccaacaacccgagggtgtcgactgactgctctggcctggtgctgcagaccggggcttggtatggaggtcgcaccgactgggtcggaaaccgttacggctcaaccgaatcgttccggctcgaccacaagatcgtctacgacctagggttcaagcggatgccccgaggcgggccagcggccttgccgatcaagccggtgatgctcgtcgggctccagcacggaggcggcggggtctactcgcacaccgcttgcacgttgatgacgatggaccaccccggtggcccggtcaagatgtccgaccgaggcgtcgactgggagtcccacggcaaccgcaacggcgtaggcgtcgaactttacgagggcgcacgggcatggaacgaccctctgttccatgacttttggtacctggacgcagtcctcgaagacgaaggagacgatgacgaattggctgacccagttctagggaagatgatccgcgagatccacgcgtgcctgttcaatcagaccgcgtcgaccagcgatctggcgacccctggtgaaggcgctatctggcagctacaccagaagatccactcgattgacggcatgctccacccgatccacgctgagcggcgcgctcgcgcaggcgatctcggtgagctgcaccgaatcgtgttggccgcgaagggcttgggcgtgaagcgcgacgaggtgaccaagcgggtctaccagagcatcctcgccgacatcgagcgggacaaccccgaagtacttcagcgatacatcgcagaaagaggtggcctatga

SEQ ID NO: 9 - protein, 400 aa, Artificial sequence
  TM4gp30 Lysin B
MAWVGWQLGMQGEQVKVIQQKLIAKYQWVRDRYPRLTASGVYDVNTQAAIVEFQFRAGLPVTGIADYATQVRLGAVAPAPPPRQRIMVLTFSGTSADMWTGYPADVARALDPSIFYWQPVCYGPNGIPAIFPMGSSAKSGEVEGLRLLDEKARDFDYIVLIGYSQGALPASRLMRRILSGDLQRFKSKLIAGVTFGNPMREKGHTFPGGADPGGHGLDPQCLVNTPDWWHDYAAKGDIYTVGSGSNDEKANADMTFIYQLVQGDILGMMFGTGNPLDILGLLGGLGGGLLGGLGGGLLGGGKGGLQLPSGLVLPGVQGGALTDHQRGLVEAVLALLANPFAEVPAAVKAIVSGVGFIATNPPTAPHIEYHIREAAPGVTYFQHAIDYLRQVGASVAARAA

SEQ ID NO: 10 - DNA, 1203 bp, Artificial sequence
  TM4gp30 Lysin B
atggcctgggtcggttggcagctcggcatgcagggggagcaggtcaaggtgatacagcaaaagctgatcgccaagtaccagtgggtgcgtgaccgttacccgcggctgacggccagcggcgtctatgacgtgaacacgcaggccgcgatcgtcgagtttcagttccgcgcagggcttcccgtcaccggcatagctgactatgcgacgcaggttcggctcggcgcggtggccccggcgccgccgccgcggcagcgcatcatggtgctgacgtttagcggcacctcggccgacatgtggaccggctatccggccgacgtcgcgcgtgcgctcgacccgtcgatcttctactggcagccagtgtgctacggccccaacggcatcccggcgatattcccgatgggttccagcgccaagagcggcgaggtcgaggggctgcggctgctcgacgagaaggcgcgcgatttcgactacatcgtgcttatcggatactcgcagggcgcgctgcccgcgtcgcggctcatgcggcgcatcctgtcgggcgacctgcagcggttcaagtccaagctgatcgccggtgtcacgttcggcaacccgatgcgcgagaaggggcacacgttccccggcggcgccgaccccggcgggcacggcctcgacccgcagtgcctcgtgaatacgcccgactggtggcacgactacgccgccaagggcgacatttacaccgtcggctcgggcagtaacgacgagaaggccaacgccgacatgacgttcatttaccagctcgtgcagggcgacattctcggcatgatgttcggcaccggcaacccgctcgacattctcggcctgctcggcggcctcggtggcggcctgctcggcggcctgggcggtggcctgctcggtggcggcaagggtggcctgcagttgccgagcggcctggtgctccccggcgtccagggcggcgcgctcaccgaccaccagcgcggcctcgtcgaggcggtgctggcgctgctcgctaacccgttcgccgaggttccggcggcggtcaaggcgattgtgtccggtgtcgggttcatcgccaccaacccgccgacggcgccgcacatcgagtaccacattcgcgaggctgcgcccggcgtgacgtatttccagcacgcgatcgactacctgcgccaggtcggcgcgtccgtcgccgctcgcgcggcctga

SEQ ID NO: 11 - protein, 321 aa, Artificial sequence
  Bxz2gp12 Lysin B
MPLRVGSNDANTGGLVSRWQKTMLARYAAYAKAYDGGPLRVDGYFGYDDADVQREYERRTHQVVDGEVSDADLRALGLEAAKRWLFTVHGTGQADPLGPGLPADTARAVLDKYTWQPIGNYPARAFPMWSSIMDGARELRSQIASKSGEVNLAGYSQGAVVVGQVLKHDIMDPKGSLHHRLGDVRKVVLWGNPMRQRGIAHFDEWIHPVAGPDSYGILDDRLEGLEKAPFEIRDYAHAGDMYASITDGDKDEYKIAICKIVMTATDFYRGPNSVVSQLIELGQRPLTEGIAMALAIIDTLRFFTNTAHGYNIGPAIDFLRS

SEQ ID NO: 12 - DNA, 966 bp, Artificial sequence
  Bxz2gp12 Lysin B
atgcccctgcgggtcgggtcgaacgacgccaataccggcgggctggtgagccgttggcagaagacgatgctggcccgctacgcggcctacgccaaggcgtacgacggcgggccgctgcgagtcgacggctatttcggctacgacgacgctgacgttcagcgcgagtacgaacgccgcacccaccaggtggtcgacggtgaggtcagtgacgccgacctccgggctctgggcctggaggcggcgaagcgctggctgttcacggtccacggcaccggacaggccgacccgctgggtccgggactccccgccgacacggcgcgggcggtgctcgacaagtacacctggcagcccatcggcaactaccccgctcgggcgttcccgatgtggtcctcgatcatggacggtgccagggagcttcgctcccagatcgcgtcaaagtccggtgaggtcaacctggcgggctactcgcaaggcgcggtggtcgtcggccaggtgctcaagcacgacatcatggacccgaagggcagcctgcaccacaggctcggcgatgtccgcaaggtagtgctctggggaaatcccatgcgccagaggggaatcgctcacttcgatgagtggattcacccggtggcaggcccagactcgtacggcatcctcgatgaccggctcgaagggctggagaaggcaccgttcgagatccgggactacgcgcacgctggtgacatgtacgcctccatcacggacggcgacaaggacgagtacaagatcgcgatctgcaagatcgtcatgacggcgacggacttctaccgaggcccgaactccgttgtgtcccaactgatcgagcttggacagcgtccgctcaccgagggcatcgcaatggccctggcgatcatcgacacgctgcggttcttcacgaacaccgcgcacggctacaacatcggaccagctatcgacttcctgcgtagctga

SEQ ID NO: 13 - protein, 254 aa, Artificial sequence
  D29gp12 Lysin B
MSKPWLFTVHGTGQPDPLGPGLPADTARDVLDIYRWQPIGNYPAAAFPMWPSVEKGVAELILQIELKLDADPYADFAMAGYSQGAIVVGQVLKHHILPPTGRLHRFLHRLKKVIFWGNPMRQKGFAHSDEWIHPVAAPDTLGILEDRLENLEQYGFEVRDYAHDGDMYASIKEDDLHEYEVAIGRIVMKASGFIGGRDSVVAQLIELGQRPITEGIALAGAIIDALTFFARSRMGDKWPHLYNRYPAVEFLRQI

SEQ ID NO: 14 - DNA, 765 bp, Artificial sequence
  D29gp12 Lysin B
atgagcaagccctggctgttcaccgttcacggcacgggccagcccgatcccctcgggcctggcctgcctgccgatacggcacgcgacgtacttgacatctaccggtggcagcccatcggcaactaccccgctgcggccttcccgatgtggccgtcggtcgagaagggtgtcgccgagctgatcctgcagatcgagctgaagctggacgcggacccctacgcggacttcgcgatggcgggttactcgcagggagccatcgtggttggccaggtgctcaagcaccacatcctgcctccgacgggcaggctccacaggttcctgcaccggctcaagaaggtcatcttctggggtaatcccatgcggcagaagggctttgcccactctgacgagtggatccacccggtcgctgcccctgacaccctcggaatcctcgaggaccggctcgaaaacctggagcagtacggcttcgaggtccgcgactacgcccacgacggtgacatgtacgcctccatcaaagaggacgacctgcacgaatacgaggtcgccatcggccggatcgtgatgaaggccagcggcttcatcggtggccgggactccgtggtagcccagctcatcgagcttggccagcgtccgatcaccgagggaattgcgttggcgggagccatcatcgacgccctcacgttcttcgcccgctctcgtatgggcgacaagtggccgcacctctacaaccgctacccggcggtcgagttcctacgacagatctga

SEQ ID NO: 15 - protein, 254 aa, Artificial sequence
  L5gp12 Lysin B
MSKPWLFTVHGTGQPDPLGPGLPADTARDVLDIYRWQPIGNYPAAAFPMWPSVEKGVAELILQIELKLDADPYADFALAGYSQGAIVVGQVLKHHIINPRGRLHRFLHRLRKVIFWGNPMRQKGFAHTDEWIHQVAASDTMGILEDRLENLEQYGFEVRDYAHDGDMYASIKEDDMHEYEVAIGRIVMSARRFIGGKDSVIAQLIELGQRPIWEGIAMARAIIDALTFFAKSTQGPSWPHLYNRFPAVEFLRRI

SEQ ID NO: 16 - DNA, 765 bp, Artificial sequence
  L5gp12 Lysin B
atgagcaagccctggctgttcaccgtccacggcacaggccagcccgacccgctcgggcctggtctgcctgccgataccgcacgggacgtacttgacatctaccggtggcagcccatcggcaactacccggcagcggcgttcccgatgtggccgtcggtcgaaaagggtgtcgctgagctgatcctgcagatcgagctgaagctggacgcagatccgtacgcggacttcgcgctggccggctactcgcagggagccatcgtggtgggccaggtgctcaagcaccacatcatcaacccgagaggtcgactgcaccggttcctgcaccggctcaggaaggtcatcttctggggtaatccgatgcggcagaagggctttgcccacaccgacgagtggattcaccaggtcgctgcctcggacacgatgggcatcctcgaggaccgactggagaacctcgagcagtacggctttgaggtccgcgactacgcgcacgacggcgacatgtacgcctccatcaaggaggacgacatgcacgagtacgaggtggccattggccgaatcgtgatgagcgctaggcgattcatcggaggtaaggactccgtcatcgcccagctcatcgagcttggacagcgtccgatctgggagggaatcgcgatggccagagccatcatcgacgccctcacgttcttcgccaagtcgacccaaggcccgagctggccgcatttgtacaaccgcttcccggcggtcgagttcctacgacgaatctga

SEQ ID NO: 17 - protein, 37 aa, Artificial sequence
  LL-37
LLGDFFRKSKEKIGKEFKRIVQRIKDFLRNLVPRTES

SEQ ID NO: 18 - DNA, 111 bp, Artificial sequence
  LL-37
ttattgggtgatttctttcggaagagcaaagaaaagataggaaaggagtttaaacgaattgttcaacgtatcaaagacttcctaaggaatcttgtaccaagaacagaaagt

SEQ ID NO: 19 - protein, 30 aa, Artificial sequence
  alpha-defensin
DCYCRIPACIAGERRYGTCIYQGRLWAFCC

SEQ ID NO: 20 - DNA, 90 bp, Artificial sequence
  alpha-defensin
gattgttattgtagaataccagcatgcattgcgggagaacgtaggtacggaacatgcatctatcaaggtcgattatgggctttttgttgc

SEQ ID NO: 21 - protein, 38 aa, Artificial sequence
  beta-defensin
NPVSCVRNKGICVPIRCPGSMKQIGTCVGRAVKCCRKK

SEQ ID NO: 22 - DNA, 114 bp, Artificial sequence
  beta-defensin
aatccagtaagctgtgttcgtaataagggtatttgcgttccaatacgatgcccaggaagtatgaaacaaatcggtacatgcgtaggaagagcagtaaagtgttgtaggaaaaaa

SEQ ID NO: 23 - protein, 25 aa, Artificial sequence
  Hepcidin
DTHFPICIFCCGCCHRSKCGMCCKT

SEQ ID NO: 24 - DNA, 75 bp, Artificial sequence
  Hepcidin
gatacacactttccaatatgcattttctgttgcggttgctgtcatagaagtaaatgtggaatgtgctgtaagaca

SEQ ID NO: 25 - protein, 27 aa, Artificial sequence
  NK-2
KILRGVCKKIMRTFLRRISKDILTGKK

SEQ ID NO: 26 - DNA, 81 bp, Artificial sequence
  NK-2
aaaatcttacgaggtgtatgtaaaaagattatgagaacatttttgcgtaggataagtaaagatatactaacaggaaagaag

SEQ ID NO: 27 - protein, 24 aa, Artificial sequence
  Ci-MAM-A24
WRSLGRTLLRLSHALKPLARRSGW

SEQ ID NO: 28 - DNA, 72 bp, Artificial sequence
  Ci-MAM-A24
tggcgaagtttaggaagaacactacttcggttgagccatgcattgaaaccattagctaggcgtagtggttgg

SEQ ID NO: 29 - protein, 588 aa, Artificial sequence
  TM4gp29/LL-37
MSFTRFLQDDPLLTREQVMAELIRVADELNMPDKRGACVIAGMTISQEVGVKDNDPPFERRFWCPANRADPESFNYPHDSESNDGRSVGYFQQQKGPNGELWWGTTASEMNLHSAATQFMTRLKAAGYNASNAQAANDSAQAIQRSGVPQAYKQWWDDINRLYDKVKGSGGGPAPAPKPPQSGPWTGDPVWLADVLRAEGLNVVELPGWLDRGHGDMGRLWGVVCHHTGSDNTPSSEIAFHPSLGLCSQIHLARNGTVTLCGVGIAWHAGVGSYPGLPEDNANAVTIGIEAQNSGTYDGAPHRTNWPDAQYDAYVKCCAAICRRLGVRADHVISHKEWAGRKQGKWDPGAIDMNIFRADVQRRIDAHQPNGEDDFMAALSADEQREVLNLLRVLADRRFVSRSPFRHLGEGPSETVAGFGLNTDGLNHAQYTIELARLGDPTHLALLREVASAEGDSRYPDRQYDAKLAKRVLAEIEGAATAPAKPSTPSAPTEPAPEAPTPPVKAACALSAAGCVVAGSTSGGGCALSTDGTGKCVVTAATDGGAAGSGSLLGDFFRKSKEKIGKEFKRIVQRIKDFLRNLVPRTES

SEQ ID NO: 30 - DNA, 1764 bp, Artificial sequence
  TM4gp29/LL-37
atgtctttcaccaggtttttacaggacgatccactgcttacccgcgaacaggtcatggctgaattgatccgagtcgcagacgagttgaatatgcctgataaaagaggtgcttgtgtaatcgctggcatgacgatttcccaagaggttggtgtcaaagataatgacccgccttttgagaggcggttctggtgcccagcaaatagggcggatccagagtcgttcaactacccccatgattcggagtcgaatgacgggagatctgtggggtacttccaacagcaaaagggtcccaacggggagctttggtggggaacgactgcgtccgaaatgaatttacatagtgcagctacacagttcatgactcgtctaaaagcggctggttataacgcatctaatgcacaggctgcgaacgatagcgctcaagctatccagcgtagcggggtgccccaagcatataagcaatggtgggatgacattaaccggttatacgacaaggtgaaaggttcaggtggaggcccggcacccgccccgaagccgccacaaagcggaccgtggactggtgacccagtgtggttagcagacgtgttgcgtgctgaaggtctaaacgtagtggagctgccggggtggttggatcggggacatggcgatatgggaaggctttggggagtagtctgtcatcacacagggagtgataatacgcccagctcggagatagccttccatccctccttagggctatgtagtcaaattcacctcgcgcgtaatggcactgttactctatgtggagtcggcatagcgtggcatgccggcgtaggatcgtatcctggtttgccagaggacaatgctaacgcagtaacaatagggatagaagcgcaaaacagtggaacgtacgacggcgcccctcacagaacaaactggcccgacgcacaatacgatgcatatgtcaaatgctgtgcggctatttgccgaagattgggcgtccgcgcggatcatgtcatttcacacaaggaatgggccggcagaaagcagggcaaatgggatcctggagcaatcgatatgaacatatttcgtgccgatgttcagcgacggatcgacgcccaccagccaaatggtgaagacgattttatggctgcattatcggcggatgaacagagggaggtactgaatctccttcgagtgcttgccgacaggcgatttgtgtcccgctccccgtttagacacttaggtgagggacctagcgaaaccgttgccggctttgggctaaacactgatggcctgaatcacgcacaatatacgatagagttggcccgactcggcgacccaacccatctggcgctgctccgggaagttgcttcagccgaaggagactctcggtatcccgatcgccagtacgacgctaaactagctaaacgcgtgctcgctgagatagaaggtgcagccaccgcccctgcgaaacctagcaccccgtcagcacccacagaaccagcgccagaagcgcctaccccgcctgttaaggctgcctgtgcacttagtgccgcgggatgcgttgtagcagggtctacaagtggaggtgggtgcgcgctcagtacagatgggacagggaagtgcgtagttactgccgcgacggacggtggagccgctgggtctgggtcactccttggcgacttctttcgtaagtcaaaggagaaaattggtaaagaatttaaacgaattgttcaaagaatcaaggacttcctaaggaacctggtaccgcgcacggagtcc

SEQ ID NO: 31 - protein, 584 aa, Artificial sequence
  TM4gp29/LL-37
MSFTRFLQDDPLLTREQVMAELIRVADELNMPDKRGACVIAGMTISQEVGVKDNDPPFERRFWCPANRADPESFNYPHDSESNDGRSVGYFQQQKGPNGELWWGTTASEMNLHSAATQFMTRLKAAGYNASNAQAANDSAQAIQRSGVPQAYKQWWDDINRLYDKVKGSGGGPAPAPKPPQSGPWTGDPVWLADVLRAEGLNVVELPGWLDRGHGDMGRLWGVVCHHTGSDNTPSSEIAFHPSLGLCSQIHLARNGTVTLCGVGIAWHAGVGSYPGLPEDNANAVTIGIEAQNSGTYDGAPHRTNWPDAQYDAYVKCCAAICRRLGVRADHVISHKEWAGRKQGKWDPGAIDMNIFRADVQRRIDAHQPNGEDDFMAALSADEQREVLNLLRVLADRRFVSRSPFRHLGEGPSETVAGFGLNTDGLNHAQYTIELARLGDPTHLALLREVASAEGDSRYPDRQYDAKLAKRVLAEIEGAATAPAKPSTPSAPTEPAPEAPTPPVKAACALSAAGCVVAGSTSGGGCALSTDGTGKCVVTAATDGGAALLGDFFRKSKEKIGKEFKRIVQRIKDFLRNLVPRTES

SEQ ID NO: 32 - DNA, 1752 bp, Artificial sequence
  TM4gp29/LL-37
atgtcctttacgcgatttctccaagatgaccccctcctaaccagggagcaagtcatggctgaactgataagggtcgcggatgagctcaacatgccagataaaagaggtgcgtgcgtaattgctgggatgactatttcgcaggaagtaggcgtcaaggataatgatcctccgtttgaaagacgtttctggtgtccggcgaaccgagcggacccggagtcctttaactatccacacgatagtgagtcaaacgacggacgttcggtcggctactttcagcaacagaaaggaccaaacggggagctctggtggggaacaacggcatctgaaatgaacttacattctgctgccacacagttcatgacgcgacttaaggcagctggatataatgcatctaatgcccaagccgcaaatgactccgcacaagcaattcagcgttcaggcgttcctcaagcgtacaagcaatggtgggacgatattaatcgtctttatgacaaagtgaagggttctggtggcgggcctgcccctgctccaaagccgcctcagtcagggccctggaccggggacccagtttggctcgctgacgtactgagagcagaggggttaaacgtagttgaattgcctggctggctagatcggggacatggggatatgggacgactgtggggtgttgtatgtcatcacacaggttcggacaacacccccagctcagaaatcgcctttcatccgagtttaggtctttgttctcagatccacctcgcaaggaatggcaccgtaacgctgtgcggagttgggatcgcctggcacgcgggtgtaggtagttatccgggtctaccagaagacaacgcaaacgcagttactatagggatagaagcccagaattcaggtacatatgatggagcaccgcatcgcactaattggcctgatgcacaatacgacgcatatgtcaagtgctgtgcggctatctgcaggcgacttggagtgcgcgcggatcatgtgattagtcacaaagaatgggctggaaggaagcaaggcaaatgggatccgggagccatagacatgaatatatttcgggcagatgtacagcgtagaatcgatgcccaccaacccaacggcgaggatgactttatggcggctttgagtgccgacgaacagagagaggttttgaacctattacgcgtgttagccgacaggagattcgtgagccggagccccttccgccatctgggagaaggtccgtcggaaaccgttgcagggttcgggttgaatacagatggactgaatcatgcgcaatacacaatagagctagctagacttggtgaccccactcacctcgctttgttacgcgaagtcgcaagcgcggagggcgacagccgttaccccgacaggcagtacgatgccaaattggctaaacgggtcctggccgaaatagagggcgcggctactgctcctgcaaaaccttccacaccatcggcgccaacagagccagcgccggaggcccctacgcccccagtgaaagcggcctgcgctttaagtgctgccggttgtgttgtagctggctccacgtctggaggcggttgtgcgttgagcactgatggcaccggaaagtgcgtggtcacggcggctaccgacgggggcgccgcactacttggggatttctttcggaagtcaaaggagaaaatcgggaaagaattcaagcgcattgtgcaacgaattaaagacttcctacggaatcttgtgccccgaactgagtcg

SEQ ID NO: 33 - protein, 544 aa, Artificial sequence
  Bzx2gp11/alpha-defensin
MTEKVLPYDRSIVTQETGWWCGPAATQVVLNSRGIIVPEATLAAEIEAIENPGRGDDRDGTDYVGLIEQVLDRRVPQARYTSVYLTNDPPTQAQKDRLWEHIVRSINAGYGVVMNWVAPPSNKPRGVKGSVSPRYSGGTTYHYVACMGYDDTPGARAVWIADSGFQPQGYWISFDQCATLIPPKGYAYADAAPAAPAPAPTPVVDAAPILARAAGISEAKAREILPTMRDGLKQADCTTVNRIAMFIAQTGHESDDFRATEEYANGPLDQERWIYKGRTWIQITWREHYARFGKWCFDRGLVTDPDVFVKNPRALADLKWAGIGAAWYWTVERPDINALCDRRDIETVSRRINGTNPNTGRANHIEERIARWNRALAVGDDLLQLIREEEDGFLSALTPAEQRALYNEIMKKGPTRSFMAEDQNQIETLLGFVYNIDGNIWNDAVTRAYLFDVPLAVEYVERVARDGVHPKSWAFQQLDGKGERWLAKFGQEYCKGLIRFKKKLNDLLEPYGENDCYCRIPACIAGERRYGTCIYQGRLWAFCC

SEQ ID NO: 34 - DNA, 1632 bp, Artificial sequence
  Bxz2gp11/alpha-defensin
atgaccgagaaggtactgccctatgacagaagtatagtcacgcaagagacgggctggtggtgcggcccggcggccacacaggttgtccttaactcgcggggcataattgttccagaagctaccttagccgcggaaatagaggcgatagaaaacccaggacgtggtgatgaccgtgatggtacagattacgtggggttgattgaacaggttctagaccgcagggttcctcaagctcgatataccagtgtctacctaactaacgatccgccaactcaagcacagaaagatcgactctgggagcacatagtacgaagcataaatgcaggatacggagtagttatgaattgggttgctccgccatcgaataagcctaggggggtaaaggggagcgtgtctcctcgctattctggaggtaccacataccattacgtagcgtgtatgggctacgatgacactcccggtgcccgggctgtttggatagcagattccggattccagccccaaggttattggatttcattcgatcaatgcgctacgctaatccctccaaaagggtatgcatacgccgatgccgcgccggcagctcccgccccggcgcctacaccagtagtggatgcggctccgatattggcaagagctgcgggtatatccgaagcaaaagcgagagagatacttccaacgatgagggatggtctaaaacaagctgactgcacaactgtcaataggatcgcgatgttcatcgcacaaactggtcacgagtcagacgattttagggcaacagaagagtacgccaacgggcctcttgaccaggaacggtggatttataaaggaagaacttggattcagatcacatggcgcgaacactacgccaggttcggcaagtggtgcttcgatcgcggcctcgtgacagaccctgatgtctttgtgaaaaatccacgggcgctggccgacctcaagtgggccggaatcggagcggcatggtattggacggtagaacggccggacattaacgcactctgtgaccgccgtgacattgaaacggtatcacgtcgaatcaatgggaccaatcctaacacgggcagagccaatcatattgaagagcgtatagctaggtggaaccgagcattggctgtgggcgacgatctactgcagttgatcagagaagaggaagacgggtttttgagcgctctaactcccgctgagcaacgcgcgctctataacgaaattatgaagaaagggccaactcgaagttttatggccgaggatcaaaaccagattgaaaccttactgggatttgtctacaatatagacggtaacatttggaatgatgccgtgaccagggcttacttattcgatgtgccccttgcagttgagtatgtcgagcgagtcgcacgggacggggtacatcccaagtcttgggcctttcagcaactggatggaaaaggcgagagatggttagctaaatttggacaggagtattgtaagggtttaatcagatttaagaaaaagcttaatgaccttttggaaccgtacggtgaaaacgactgctattgtcgtatccccgcgtgtattgcaggggagcggcgttatggcacatgcatctatcaagggcgattatgggcgttctgttgt

SEQ ID NO: 35 - protein, 544 aa, Artificial sequence
  alpha-defensin/Bzx2gp11
DCYCRIPACIAGERRYGTCIYQGRLWAFCCMTEKVLPYDRSIVTQETGWWCGPAATQVVLNSRGIIVPEATLAAEIEAIENPGRGDDRDGTDYVGLIEQVLDRRVPQARYTSVYLTNDPPTQAQKDRLWEHIVRSINAGYGVVMNWVAPPSNKPRGVKGSVSPRYSGGTTYHYVACMGYDDTPGARAVWIADSGFQPQGYWISFDQCATLIPPKGYAYADAAPAAPAPAPTPVVDAAPILARAAGISEAKAREILPTMRDGLKQADCTTVNRIAMFIAQTGHESDDFRATEEYANGPLDQERWIYKGRTWIQITWREHYARFGKWCFDRGLVTDPDVFVKNPRALADLKWAGIGAAWYWTVERPDINALCDRRDIETVSRRINGTNPNTGRANHIEERIARWNRALAVGDDLLQLIREEEDGFLSALTPAEQRALYNEIMKKGPTRSFMAEDQNQIETLLGFVYNIDGNIWNDAVTRAYLFDVPLAVEYVERVARDGVHPKSWAFQQLDGKGERWLAKFGQEYCKGLIRFKKKLNDLLEPYGEN

SEQ ID NO: 36 - DNA, 1632 bp, Artificial sequence
  alpha-defensin/Bzx2gp11
gattgctactgccggatacccgcgtgcattgcaggggagcggagatacggaacatgtatttatcagggccgcctttgggcattttgttgcatgactgagaaggtgctaccgtacgataggtcaatcgttacccaggaaactggatggtggtgtgggccagcggccacacaggttgtgcttaacagcaggggaatcatagtgccggaggcaactctcgcagcggaaatagaagcaattgaaaaccctgggcgtggtgatgacagggacggaacagattacgttggcctaatcgagcaagtcctggaccgcagagtaccgcaggcgcgatatacatccgtgtatctcacgaacgatcctcccacgcaagcgcagaaggataggttgtgggaacatatcgtccgaagtatcaatgctggctacggcgtggttatgaattgggtagccccgccatctaataagccacgaggtgtaaaaggatctgtgagtccgcgttactcaggcgggacgacatatcattacgtcgcgtgtatgggatatgatgacacccctggagctcgagcagtttggattgcggattccggtttccagcctcaagggtactggataagctttgaccaatgcgctactcttatacctccaaagggttacgcatatgcagacgctgcgcccgccgctcctgcacccgctcccacacccgttgtagacgccgcgcccattttggcccgtgcggccggaataagcgaggcaaaagctagggaaatactaccaactatgagggatggactaaaacaagccgattgtacgactgtcaaccgcatcgcgatgtttattgctcagacgggtcacgaatcagatgactttcgagctacggaggaatatgcaaacggtccattagatcaagagcgatggatatataaaggccgcacctggattcagataacatggagagaacactatgctcggttcggtaaatggtgtttcgatcggggattagtgactgatccggatgtattcgtcaagaaccccagagccttggccgacttgaaatgggccggtataggcgccgcgtggtattggaccgtcgagagaccagacattaatgcactctgtgaccggcgcgacattgagaccgtttctcgtaggatcaatggaacaaatccaaacacgggtcgtgctaatcatattgaggaacggatagcgcgttggaaccgggccctcgctgttggggatgaccttctacagttaattcgagaagaggaagacgggtttttatcggcacttactcctgctgaacaaagagcattatataatgaaatcatgaaaaagggcccaacccgaagtttcatggctgaagatcaaaatcaaattgagacactgttagggtttgtatataacatcgacggtaatatatggaatgacgcagtaacccgtgcctacctatttgatgtacctttggccgtcgagtacgtagagagagtggcgcgcgacggggtccacccgaagtcgtgggctttccagcaactggatggtaaaggcgagaggtggctggcgaagttcgggcaagaatattgcaagggcttgatcagatttaaaaagaaactcaacgacctgcttgagccgtacggggaaaac

SEQ ID NO: 37 - protein, 574 aa, Artificial sequence
  alpha-defensin/Bzx2gp11/alpha-defensin
DCYCRIPACIAGERRYGTCIYQGRLWAFCCMTEKVLPYDRSIVTQETGWWCGPAATQVVLNSRGIIVPEATLAAEIEAIENPGRGDDRDGTDYVGLIEQVLDRRVPQARYTSVYLTNDPPTQAQKDRLWEHIVRSINAGYGVVMNWVAPPSNKPRGVKGSVSPRYSGGTTYHYVACMGYDDTPGARAVWIADSGFQPQGYWISFDQCATLIPPKGYAYADAAPAAPAPAPTPVVDAAPILARAAGISEAKAREILPTMRDGLKQADCTTVNRIAMFIAQTGHESDDFRATEEYANGPLDQERWIYKGRTWIQITWREHYARFGKWCFDRGLVTDPDVFVKNPRALADLKWAGIGAAWYWTVERPDINALCDRRDIETVSRRINGTNPNTGRANHIEERIARWNRALAVGDDLLQLIREEEDGFLSALTPAEQRALYNEIMKKGPTRSFMAEDQNQIETLLGFVYNIDGNIWNDAVTRAYLFDVPLAVEYVERVARDGVHPKSWAFQQLDGKGERWLAKFGQEYCKGLIRFKKKLNDLLEPYGENDCYCRIPACIAGERRYGTCIYQGRLWAFCC

SEQ ID NO: 38 - DNA, 1722 bp, Artificial sequence
  alpha-defensin/Bzx2gp11/alpha-defensin
gattgctactgtaggattcctgcgtgcatagcaggagaaagacgatacggtacttgcatttatcagggtcgtctgtgggcgttctgttgcatgacagagaaagtacttccctatgaccggtccatcgtaacgcaggaaactggctggtggtgcggcccagccgcgacgcaagtagtcctgaatagtagagggattatagtacctgaagcaacactcgctgcagagatcgaggcaatagagaacccaggacgtggtgatgaccgcgacggtactgattacgttgggttgatcgaacaggtcttggaccggagagtaccgcaagctaggtatacttcggtgtacctgacaaacgatccgcctacacaggcacaaaaagacaggctatgggagcacattgttcgatcaataaatgccggttacggagtggtcatgaactgggtggctccgccttctaacaaacccagaggagtcaaagggagcgtttcaccccggtattctggcggtactacatatcattacgttgcctgtatgggatacgatgacacacccggtgcccgcgcggtgtggattgccgactccggttttcagcctcaaggatattggatatcattcgatcagtgtgccaccttgatcccaccgaagggctacgcctatgccgatgcggctcccgctgcgccagcgccagcacctactcccgtggttgacgcggcacctatacttgcccgcgctgcaggtattagcgaggccaaagcgcgcgagatacttccgactatgagggatgggcttaagcaagcagactgtacaacggtgaaccgaattgcgatgtttatagcccaaaccgggcacgagagtgatgactttcgagccactgaagagtatgctaatgggcccctagatcaggaacgatggatatataagggtcgaacgtggatccagattacatggagagaacattatgcgagattcggcaaatggtgctttgaccgtggactcgtaaccgaccccgacgtatttgtgaagaatcctcgcgccttagctgatctcaagtgggcagggattggcgctgcgtggtactggacggtagaacggccggatattaatgctttatgtgacaggcgtgacatagagacagtttcgcgaaggattaacgggaccaacccaaatacgggaagagcgaaccacattgaggaaagaatcgcgcgatggaatagggcactcgcagtcggcgacgatttacttcagttaatacgtgaggaagaggacggattcctgagcgctctgacgccggcagagcaaagggcattgtataacgaaataatgaaaaagggtccaacccggtctttcatggctgaagatcagaatcaaatcgagaccctattaggctttgtatataacatcgacgggaatatatggaacgatgctgtgacgagagcgtacttattcgacgtcccactcgctgtcgaatatgttgaacgcgttgcccgtgatggtgtccatcctaaaagttgggcttttcaacagcttgatggcaagggcgaacgatggttggctaagtttggacaagaatactgtaagggcctaattaggttcaagaaaaaattgaatgatctactggagccgtacggggaaaatgattgttactgccggatcccagcctgtatcgcaggagagcgccggtatgggacctgcatctaccaaggacgtctatgggcattctgctgt

SEQ ID NO: 39 - protein, 330 aa, Artificial sequence
  beta-defensin/L5gp10
NPVSCVRNKGICVPIRCPGSMKQIGTCVGRAVKCCRKKMTFTVTRERAQWVHDMARARDGLPYAYGGAFTNNPRVSTDCSGLVLQTGAWYGGRTDWVGNRYGSTESFRLDHKIVYDLGFKRMPRGGPAALPIKPVMLVGLQHGGGGVYSHTACTLMTMDHPGGPVKMSDRGVDWESHGNRNGVGVELYEGARAWNDPLFHDFWYLDAVLEDEGDDDELADPVLGKMIREIHACLFNQTASTSDLATPGEGAIWQLHQKIHSIDGMLHPIHAERRARAGDLGELHRIVLAAKGLGVKRDEVTKRVYQSILADIERDNPEVLQRYIAERGGL

SEQ ID NO: 40 - DNA, 990 bp, Artificial sequence
  beta-defensin/L5gp10
aatcccgttagttgtgttcgaaataagggcatctgcgtgccaatccgatgcccgggttccatgaagcagattgggacatgcgtcggtcgtgcggtaaaatgttgcaggaagaaaatgacgtttacggtaacccgggagagagcccaatgggtgcatgatatggctagggcacgggatgggctaccttacgcgtacggtggagcgtttaccaacaatccgcgagtcagcacggactgttcggggttagtgttgcagaccggcgcatggtacggcggaagaacagattgggtgggtaacaggtatggtagtactgagtcgttccggttagatcataaaattgtttatgacttaggatttaagcgtatgcctcgcggtggacccgcggcattgcctatcaaaccagtaatgctggtcggccttcaacatgggggcggaggcgtttacagtcacactgcgtgtacacttatgacaatggaccaccccgggggcccagtaaaaatgtctgatcgtggagtcgactgggagtctcacggtaacagaaatggtgtaggggtcgagttgtatgagggagctcgcgcctggaatgacccgctgtttcatgatttctggtatctagacgcggtattagaagatgaaggagacgatgacgaacttgctgatcctgtgctagggaagatgattcgggaaatccacgcctgtctcttcaaccaaacggcatccactagcgatctggcaactccgggagagggggcaatatggcaattacaccagaaaatccactcaatagatggtatgttgcatccaatacatgcagagcgccgtgcccgagctggagacctcggtgaactccatcgcattgttttggctgccaagggcctcggggtaaagagggacgaagttaccaaacgagtgtatcagtcaattctagctgatatagagagagacaaccccgaagtcctgcaaagatacatagccgaaagggggggcctt

SEQ ID NO: 41 - protein, 359 aa, Artificial sequence
  beta-defensin/Hepcidin/L5gp10
NPVSCVRNKGICVPIRCPGSMKQIGTCVGRAVKCCRKKGAGADTHFPICIFCCGCCHRSKCGMCCKTMTFTVTRERAQWVHDMARARDGLPYAYGGAFTNNPRVSTDCSGLVLQTGAWYGGRTDWVGNRYGSTESFRLDHKIVYDLGFKRMPRGGPAALPIKPVMLVGLQHGGGGVYSHTACTLMTMDHPGGPVKMSDRGVDWESHGNRNGVGVELYEGARAWNDPLFHDFWYLDAVLEDEGDDDELADPVLGKMIREIHACLFNQTASTSDLATPGEGAIWQLHQKIHSIDGMLHPIHAERRARAGDLGELHRIVLAAKGLGVKRDEVTKRVYQSILADIERDNPEVLQRYIAERGGL

SEQ ID NO: 42 - DNA, 1077 bp, Artificial sequence
  beta-defensin/Hepcidin/L5gp10
aatcctgtgagttgtgttcgaaataagggtatttgtgtcccgatacgttgccccggttcgatgaagcaaattggtacttgcgtcggacgtgccgtaaagtgttgccgaaagaaaggtgcaggcgccgatacgcattttccgatctgtattttctgctgtggatgttgccataggtctaaatgcggaatgtgttgcaaaactatgacatttacagtaacacgagaacgggcacagtgggtccacgatatggcaagggctcgagacgggctaccatatgcatatggcggggcattcacgaataaccccagggtgagcaccgactgctccggtcttgtactgcaaactggggcctggtacggcggacgaactgactgggtaggtaaccgttatggtagcacggagtccttccgtctcgaccacaaaatagtctacgacctgggttttaaaagaatgccaagagggggacccgcggctcttccaataaagcccgttatgttagttggattacagcacgggggagggggcgtatattcacacacagcctgtacgttgatgaccatggatcatcctgggggtccggttaagatgagtgacaggggcgtcgattgggagtcgcacggcaaccgcaacggcgtaggcgtggagttgtacgagggagctagggcttggaatgacccactcttccacgatttttggtacctagatgcagtgcttgaagacgagggagatgacgatgaactagccgacccagtgttggggaaaatgatacgcgaaatacatgcctgtctatttaaccagaccgcttctacttcagatttggcgacaccgggcgaaggggcgatctggcaattacatcaaaaaattcatagtattgacgggatgttacatcctatccacgcggaacggagagcacgggctggtgatctgggcgaattgcatcggatagttcttgcggctaaggggttaggagttaaacgcgacgaggtcaccaagagagtataccaaagcatcctggcggatatcgagcgcgataatcctgaagtgctccagagatatattgcggagcgtggaggtctc

SEQ ID NO: 43 - protein, 441 aa, Artificial sequence
  TM4gp30/LL-37
MAWVGWQLGMQGEQVKVIQQKLIAKYQWVRDRYPRLTASGVYDVNTQAAIVEFQFRAGLPVTGIADYATQVRLGAVAPAPPPRQRIMVLTFSGTSADMWTGYPADVARALDPSIFYWQPVCYGPNGIPAIFPMGSSAKSGEVEGLRLLDEKARDFDYIVLIGYSQGALPASRLMRRILSGDLQRFKSKLIAGVTFGNPMREKGHTFPGGADPGGHGLDPQCLVNTPDWWHDYAAKGDIYTVGSGSNDEKANADMTFIYQLVQGDILGMMFGTGNPLDILGLLGGLGGGLLGGLGGGLLGGGKGGLQLPSGLVLPGVQGGALTDHQRGLVEAVLALLANPFAEVPAAVKAIVSGVGFIATNPPTAPHIEYHIREAAPGVTYFQHAIDYLRQVGASVAARAAGSGSLLGDFFRKSKEKIGKEFKRIVQRIKDFLRNLVPRTES

SEQ ID NO: 44 - DNA, 1323 bp, Artificial sequence
  TM4gp30/LL-37
atggcctgggtaggctggcaattaggtatgcaaggcgagcaagtaaaggtcatacaacagaagctgatcgctaagtaccaatgggtgagggaccgctatcctaggctaacggcatctggagtctatgatgtgaatactcaagcggctattgtcgaattccaatttcgagccggtttaccagttaccgggattgcggattacgccactcaagtacgcttaggagccgtagcgcccgcaccacctccgagacagcgcatcatggtacttacattcagcggcacatcagctgacatgtggaccggctacccagctgacgttgcgcgtgctttggacccttccatattttactggcagcccgtttgttatggtcctaatggcattccagcgatatttcccatggggagctccgcaaagtccggggaagtagaaggactccggttactagacgagaaagccagggatttcgactatatcgttctgatcggttactctcagggcgctctccctgcgtcacgtctgatgagacgtatactttcgggcgacctgcagagattcaagagtaagttgattgccggagtgacatttggaaaccccatgcgcgagaaagggcatacgtttccgggtggagcggacccgggtgggcatggtctcgatccccagtgcctagttaacacaccggattggtggcacgattatgccgcaaaaggtgacatatatactgttgggtcgggcagtaatgatgagaaagctaacgcagacatgacttttatctatcagctcgtgcaaggggatatactcgggatgatgttcggtacggggaatccgttggatattctaggtctcttagggggtttagggggtggacttctaggagggctcggaggcggtctattaggtggcggaaagggaggccttcagcttccaagcgggttggtcttaccaggcgtccaaggaggcgccctgaccgatcatcagcggggcttggtagaggctgtcctagcacttttggcgaaccctttcgcggaggtacccgctgccgtgaaggccattgtgagcggtgtgggtttcatcgcaacgaaccctccgaccgcaccacacatagaataccatataagagaagcggctcccggagtcacttactttcaacacgccatcgattatttgcgacaggtgggagcaagtgttgcagctagggcggcagggtcaggatctcttctgggggactttttccgaaaatcgaaagaaaagattggaaaagaatttaaacggatagttcagcggattaaagattttttgcgtaatctggtcccgcgaacagagagt

SEQ ID NO: 45 - protein, 437 aa, Artificial sequence
  TM4gp30/LL-37
MAWVGWQLGMQGEQVKVIQQKLIAKYQWVRDRYPRLTASGVYDVNTQAAIVEFQFRAGLPVTGIADYATQVRLGAVAPAPPPRQRIMVLTFSGTSADMWTGYPADVARALDPSIFYWQPVCYGPNGIPAIFPMGSSAKSGEVEGLRLLDEKARDFDYIVLIGYSQGALPASRLMRRILSGDLQRFKSKLIAGVTFGNPMREKGHTFPGGADPGGHGLDPQCLVNTPDWWHDYAAKGDIYTVGSGSNDEKANADMTFIYQLVQGDILGMMFGTGNPLDILGLLGGLGGGLLGGLGGGLLGGGKGGLQLPSGLVLPGVQGGALTDHQRGLVEAVLALLANPFAEVPAAVKAIVSGVGFIATNPPTAPHIEYHIREAAPGVTYFQHAIDYLRQVGASVAARAALLGDFFRKSKEKIGKEFKRIVQRIKDFLRNLVPRTES

SEQ ID NO: 46 - DNA, 1311 bp, Artificial sequence
  TM4gp30/LL-37
atggcgtgggtcggttggcagttaggcatgcaaggagagcaggttaaggttatacaacagaagcttatcgccaaatatcagtgggtacgtgaccgctacccacgccttacagcttcaggcgtgtatgatgtaaacacacaggctgcaatagtcgagtttcaattccgagctgggctaccggtgacggggatcgcggactacgctactcaggtgcgtctcggtgcagtggcgcctgctcctccgccccgccagcggataatggtactaaccttcagtggtacatcggccgatatgtggactggttaccccgccgatgttgcccgagccttggacccctcgatattctattggcaacctgtttgctatggtccaaatggtattccagcaatctttcccatgggtagctcggcgaagagtggcgaagtggaggggcttaggttattggatgaaaaggctagagacttcgattacattgtcctaatagggtattcacagggggcactacctgctagtcgactaatgaggcgcattctgagcggcgatttacaaaggttcaaatcaaagctaatagcgggcgtaaccttcggaaacccgatgcgggaaaaaggtcacacctttcctggaggggcggaccctggcggtcatggactggatccacaatgtctcgtcaacacgcccgactggtggcatgattatgcagctaaaggagatatttacaccgttgggtccggctctaacgatgaaaaagctaacgccgacatgacattcatttaccaactggtacaaggggacatcttaggaatgatgtttggtacggggaatccgctcgatatacttgggttactgggaggtctcgggggcggactcttgggcggattgggagggggtctactcggcggtggcaagggagggcttcagttaccatctggcctggtactgccgggagtacaaggtggagcattgactgaccatcaacgtggtttagtcgaggccgtgttggctcttctcgcaaatccatttgccgaagtgcctgcagcggtcaaagccatcgtatctggcgtcgggtttattgcgactaatccgcccacagcgccacacatcgagtatcatataagggaggcggctcccggagttacttactttcaacacgccatcgactatttgagacaggttggggcctccgttgcagcgcgggcagcattattgggcgactttttccgaaagtccaaagaaaaaattggaaaggagtttaagagaattgtgcagagaataaaagattttcttcgtaatctggtcccgcggacggaaagc

SEQ ID NO: 47 - protein, 284 aa, Artificial sequence
  D29gp12/alpha-defensin
MSKPWLFTVHGTGQPDPLGPGLPADTARDVLDIYRWQPIGNYPAAAFPMWPSVEKGVAELILQIELKLDADPYADFAMAGYSQGAIVVGQVLKHHILPPTGRLHRFLHRLKKVIFWGNPMRQKGFAHSDEWIHPVAAPDTLGILEDRLENLEQYGFEVRDYAHDGDMYASIKEDDLHEYEVAIGRIVMKASGFIGGRDSVVAQLIELGQRPITEGIALAGAIIDALTFFARSRMGDKWPHLYNRYPAVEFLRQIDCYCRIPACIAGERRYGTCIYQGRLWAFCC

SEQ ID NO: 48 - DNA, 852 bp, Artificial sequence
  D29gp12/alpha-defensin
atgtctaaaccatggctcttcacggtgcatggcaccgggcaacctgatccactaggtccgggactacctgccgacacagctcgcgacgtcttagatatatatcgatggcaacccataggcaactacccggccgcggctttccctatgtggccatcggtcgagaagggtgtagcggagttaattctgcagattgaattaaagctggatgcagatccttacgctgatttcgctatggcggggtatagccagggcgcgatcgtagtgggtcaggtgctcaaacatcacattcttccgcccactggaaggctgcaccgctttcttcacagactcaagaaagttatattttggggaaatcccatgcgtcaaaagggatttgctcattccgacgaatggatccatccagttgcagccccggataccttgggaattctcgaggatagattagagaatttggaacaatacgggtttgaggtacgcgattatgcgcatgacggggacatgtatgcaagtataaaagaagatgacctacacgaatacgaggtagcgatcggacgtatagttatgaaagcatcaggctttataggtggccgggacagtgtggttgcgcagttaatagaactgggtcaaagaccgattacagaaggcattgcccttgctggtgcaatcattgacgccttgacttttttcgctagaagccgaatgggtgataagtggccacacctatataacaggtatcccgcagtcgaattcttgcgtcaaatcgactgttattgcaggatacctgcatgcatcgccggagagcggcgatacgggacgtgtatttaccaggggcggctttgggccttctgttgc

SEQ ID NO: 49 - protein, 284 aa, Artificial sequence
  alpha-defensin/D29gp12
DCYCRIPACIAGERRYGTCIYQGRLWAFCCMSKPWLFTVHGTGQPDPLGPGLPADTARDVLDIYRWQPIGNYPAAAFPMWPSVEKGVAELILQIELKLDADPYADFAMAGYSQGAIVVGQVLKHHILPPTGRLHRFLHRLKKVIFWGNPMRQKGFAHSDEWIHPVAAPDTLGILEDRLENLEQYGFEVRDYAHDGDMYASIKEDDLHEYEVAIGRIVMKASGFIGGRDSVVAQLIELGQRPITEGIALAGAIIDALTFFARSRMGDKWPHLYNRYPAVEFLRQI

SEQ ID NO: 50 - DNA, 852 bp, Artificial sequence
  alpha-defensin/D29gp12
gactgctattgtagaatacctgcctgtatcgcaggagagcgacggtacggaacctgcatctaccagggtcgcttatgggcgttctgctgtatgtccaaaccttggctctttacagtacatggaacagggcaaccggatccgttggggccgggtcttcctgcagacaccgcgagagacgtattagacatctacaggtggcaacctattggcaattacccagccgcggcattcccaatgtggccaagtgtggagaagggcgtcgctgaacttattttgcaaatagagctcaagctcgatgctgatccgtatgctgattttgctatggcaggttattcgcagggtgctatagtggtcggccaggttctaaagcatcacatccttcccccaacgggcagactacatcgttttttacacaggttgaagaaagttattttttggggaaatcccatgcgtcagaaaggttttgcacactctgacgagtggattcatccagtggcggcccccgatactctcggcatcctagaagatcgtctagaaaacttggaacagtatggtttcgaggttagggattatgcgcacgatggagacatgtatgcaagcataaaagaagatgacttacacgaatacgaagttgcaattggacgcatcgtcatgaaggcctcaggtttcataggcgggcgggacagtgtagtggctcaactgatagagttaggacaacgaccgataactgaagggatagccctggcgggggccattattgacgccctgacgttctttgctcgcagccggatgggggataaatggccccatctgtataacagataccctgcggtagagttccttcgacaaatt

SEQ ID NO: 51 - protein, 314 aa, Artificial sequence
  alpha-defensin/D29gp12/alpha-defensin
DCYCRIPACIAGERRYGTCIYQGRLWAFCCMSKPWLFTVHGTGQPDPLGPGLPADTARDVLDIYRWQPIGNYPAAAFPMWPSVEKGVAELILQIELKLDADPYADFALAGYSQGAIVVGQVLKHHIINPRGRLHRFLHRLRKVIFWGNPMRQKGFAHTDEWIHQVAASDTMGILEDRLENLEQYGFEVRDYAHDGDMYASIKEDDMHEYEVAIGRIVMSARRFIGGKDSVIAQLIELGQRPIWEGIAMARAIIDALTFFAKSTQGPSWPHLYNRFPAVEFLRRIDCYCRIPACIAGERRYGTCIYQGRLWAFCC

SEQ ID NO: 52 - DNA, 942 bp, Artificial sequence
  alpha-defensin/D29gp12/alpha-defensin
gactgctattgtcggattcctgcttgcatcgcaggtgaaagacgatatggcacatgtatatatcaaggccgcctttgggcgttttgctgtatgtctaaaccgtggctcttcacggtgcatggtacaggacaaccagacccccttggtcctggattgcctgcggatacggcacgtgatgttctggacatatatcgatggcagcccataggtaactacccagcagctgcctttccgatgtggccaagtgttgagaaaggcgtagccgaactcattttgcaaattgagctgaagctggatgcagatccctacgccgattttgcacttgccggctacagtcagggtgcaatcgtcgtaggccaggttttaaagcatcacataatcaatccaagaggacgcctccaccgtttcttacaccgcctgcgcaaggtcatcttttggggaaatccgatgagacaaaaggggttcgcccatactgacgagtggatacaccaagtagcagcgtcagataccatgggaatattggaggaccgacttgaaaacctagagcaatatggctttgaagtccgtgactacgctcatgacggggatatgtacgctagcatcaaagaggatgacatgcatgaatatgaagtggcgattgggaggattgtgatgtccgcgaggcggttcatagggggtaaagactcagttattgcacagttgatagagctaggtcagaggcccatttgggaagggatcgccatggctcgggctataatcgatgccctaactttctttgctaaatcgacccaagggcctagctggccacacctatacaataggttccctgcggtagagtttttaaggcgtatcgattgctattgccggattccggcgtgtattgcgggagaacgaagatatgggacatgtatataccagggaagattatgggctttctgttgc

SEQ ID NO: 53 - protein, 292 aa, Artificial sequence
  beta-defensin/D29gp12
NPVSCVRNKGICVPIRCPGSMKQIGTCVGRAVKCCRKKMSKPWLFTVHGTGQPDPLGPGLPADTARDVLDIYRWQPIGNYPAAAFPMWPSVEKGVAELILQIELKLDADPYADFAMAGYSQGAIVVGQVLKHHILPPTGRLHRFLHRLKKVIFWGNPMRQKGFAHSDEWIHPVAAPDTLGILEDRLENLEQYGFEVRDYAHDGDMYASIKEDDLHEYEVAIGRIVMKASGFIGGRDSVVAQLIELGQRPITEGIALAGAIIDALTFFARSRMGDKWPHLYNRYPAVEFLRQI

SEQ ID NO: 54 - DNA, 876 bp, Artificial sequence
  beta-defensin/D29gp12
aatcctgtctcttgtgtcaggaacaaagggatttgcgttcccatccgctgccctggatcgatgaagcagatcggaacatgtgtcggcagagctgtgaaatgctgtaggaaaaagatgagtaaaccatggttgttcacggtacacggcacagggcagcccgacccccttgggcccggattacctgcggataccgctcgggacgtgttagatatctaccgttggcaaccgataggtaactacccagcagcggcctttccaatgtggccttcagtagagaagggggttgctgagcttatactacagattgaactcaagcttgatgccgacccatatgctgatttcgcaatggcaggttactctcaaggtgccatcgttgtcggtcaagtgctaaaacatcacattctaccacctactggacgtcttcacaggttcttgcaccggctcaaaaaggtaatattctggggcaatccgatgcgtcagaaaggatttgctcactcagatgaatggattcatccggtagcagccccggacacgttggggatcctggaggaccgcctcgaaaacttagagcaatatggtttcgaggtcagagattacgcgcatgatggcgatatgtatgcgagcattaaggaagacgatttacatgagtatgaagttgcaatagggcgaatagttatgaaggctagtggttttatagggggccgagactccgtggtagctcagctgattgaactgggacaaagaccaataactgagggaattgcgctggccggcgcgattatcgacgccttgaccttttttgcacggagccgcatgggtgacaaatggccgcatctatacaatagatatcccgcagtggaatttctccgacaaata

SEQ ID NO: 55 - protein, 321 aa, Artificial sequence
  beta-defensin/Hepcidin/L5gp12
NPVSCVRNKGICVPIRCPGSMKQIGTCVGRAVKCCRKKGAGADTHFPICIFCCGCCHRSKCGMCCKTMSKPWLFTVHGTGQPDPLGPGLPADTARDVLDIYRWQPIGNYPAAAFPMWPSVEKGVAELILQIELKLDADPYADFALAGYSQGAIVVGQVLKHHIINPRGRLHRFLHRLRKVIFWGNPMRQKGFAHTDEWIHQVAASDTMGILEDRLENLEQYGFEVRDYAHDGDMYASIKEDDMHEYEVAIGRIVMSARRFIGGKDSVIAQLIELGQRPIWEGIAMARAIIDALTFFAKSTQGPSWPHLYNRFPAVEFLRRI

SEQ ID NO: 56 - DNA, 963 bp, Artificial sequence
  beta-defensin/Hepcidin/L5gp12
aatccggtctcctgtgttagaaacaagggaatatgcgtccctatacgatgcccaggttctatgaaacagatcggaacctgtgtaggtcgggctgtgaagtgctgtcggaaaaagggagccggcgccgacactcactttccaatatgcattttttgctgtgggtgctgtcatcgtagtaaatgcggtatgtgttgtaaaacaatgtcaaaaccgtggctgtttacagtgcatggtactgggcaacccgatccccttggcccggggttaccagcagacaccgcacgcgatgtacttgatatctatcggtggcaaccaatcggcaattatccggccgctgcgtttcccatgtggccctcggtcgagaagggggtggcggaactaatcctacaaatagagctgaagctcgacgccgacccatacgcggatttcgcactcgcaggctatagccagggagctattgttgtcggccaggtgttaaaacaccatataattaatcctcgagggcgcttgcacaggttcttacatcgtttgaggaaagtaatattctggggcaatccgatgcgccagaaggggtttgcgcacacggatgaatggattcatcaagtagccgcttctgacacaatgggtatactagaggatagacttgagaacttagaacaatatggattcgaagttagggattacgctcacgacggagacatgtatgcgtccataaaggaagacgatatgcatgagtacgaggtagcaattggccgaattgtgatgtcggctcgtagattcattggaggtaaagatagtgttattgcgcagttgatagaattgggtcagcgtcctatctgggagggaatcgccatggcgagagctatcattgacgcactgacgtttttcgcaaagtcaactcaagggcctagctggcctcacctatacaaccggttccccgccgttgaatttctccgaaggatc

SEQ ID NO: 57 - DNA, 20 bp, Artificial sequence
  primer 27f(190)
agagtttgatcctggctcag

SEQ ID NO: 58 - DNA, 22 bp, Artificial sequence
  primer 1492r(191)
tacggttaccttgttacgactt

(19) United States
(12) Patent Application Publication
(10) Pub. No.: US 2015/0165013 A1
(43) Pub. Date: Jun. 18, 2015

(54) COMPOSITION FOR USE IN MYCOBACTERIA VACCINATION

(71) Applicant: Lysando AG, Triesenberg (LI)

(72) Inventor: Stefan Miller, Regensburg (DE)

(21) Appl. No.: 14/411,770

(22) PCT Filed: Jul. 1, 2013
(86) PCT No.: PCT/EP2013/063843

§ 371 Date: Dec. 29, 2014

(30) Foreign Application Priority Data
Jun. 29, 2012 (EP) 12174472.6

(51) Int. Cl. A61K 39/04; C12N 1/20; C12N 1/06; C07K 16/18

(52) U.S. Cl. CPC A61K 39/04; C07K 16/18; C12N 1/20; C12N 1/06

(57) ABSTRACT

The present invention relates to a method for the preparation of a mycobacterial lysate comprising the steps of: a) contacting a sample comprising at least one Mycobacterium species with a composition having the activity of degrading the cell wall of a Mycobacterium species, the composition comprising: (a) a first fusion protein including (i) a first endolysin or a first domain, both having a first enzymatic activity, the enzymatic activity being at least one or more of the following: N-acetyl-b-D-muramidase (lysozyme, lytic transglycosylase), N-acetyl-b-D-glucosaminidase, N-acetylmuramoyl-L-alanine amidase, L-alanoyl-D-glutamate (LD) endopeptidase, c-D-glutamyl-meso-diaminopimelic acid (DL) peptidase, L-alanyl-D-iso-glutaminyl-meso-diaminopimelic acid (D-Ala-m-DAP) (DD) endopeptidase, or m-DAP-m-DAP (LD) endopeptidase; and (ii) at least one peptide stretch fused to the N- or C-terminus of the endolysin having the first enzymatic activity or the domain having the first enzymatic activity, wherein the peptide stretch is selected from the group consisting of synthetic amphipathic peptide, synthetic cationic peptide, synthetic polycationic peptide, synthetic hydrophobic peptide, synthetic antimicrobial peptide (AMP) or naturally occurring AMP; and (b) a second fusion protein including (i) a second endolysin or a second domain, both having a second enzymatic activity, the enzymatic activity being at least one or more of the following: lipolytic activity, cutinase, mycolarabinogalactanesterase, or alpha/beta hydrolase; and (ii) at least one peptide stretch fused to the N- or C-terminus of the endolysin having a second enzymatic activity or the domain having the second enzymatic activity, wherein the peptide stretch is selected from the group consisting of synthetic amphipathic peptide, synthetic cationic peptide, synthetic polycationic peptide, synthetic hydrophobic peptide, synthetic antimicrobial peptide (AMP) or naturally occurring AMP; b) incubating the sample for a distinct period, and c) isolating the mycobacterial lysate resulting from step b) thereby obtaining the mycobacterial lysate. Moreover, the present invention relates to the a mycobacterial lysate obtained by the method of the present invention and further to a vaccine composition comprising the mycobacterial lysate, an antibody or antibody fragment generated with the mycobacterial lysate or the vaccine, and a pharmaceutical composition comprising the antibody or antibody fragment.

The present invention relates to a method for the preparation of a mycobacterial lysate comprising the steps of: a) contacting a sample comprising at least one Mycobacterium species with a composition having the activity of degrading the cell wall of a Mycobacterium species, the composition comprising: (a) a first fusion protein including (i) a first endolysin or a first domain, both having a first enzymatic activity, the enzymatic activity being at least one or more of the following: N-acetyl-b-D-muramidase (lysozyme, lytic transglycosylase), N-acetyl-b-D-glucosaminidase, N-acetylmuramoyl-L-alanine amidase, L-alanoyl-D-glutamate (LD) endopeptidase, c-D-glutamyl-meso-diaminopimelic acid (DL) peptidase, L-alanyl-D-iso-glutaminyl-meso-diaminopimelic acid (D-Ala-m-DAP) (DD) endopeptidase, or m-DAP-m-DAP (LD) endopeptidase; and (ii) at least one peptide stretch fused to the N- or C-terminus of the endolysin having the first enzymatic activity or the domain having the first enzymatic activity, wherein the peptide stretch is selected from the group consisting of synthetic amphipathic peptide, synthetic cationic peptide, synthetic polycationic peptide, synthetic hydrophobic peptide, synthetic antimicrobial peptide (AMP) or naturally occurring AMP; and (b) a second fusion protein including (i) a second endolysin or a second domain, both having a second enzymatic activity, the enzymatic activity being at least one or more of the following: lipolytic activity, cutinase, mycolarabinogalactanesterase, or alpha/beta hydrolase; and (ii) at least one peptide stretch fused to the N- or C-terminus of the endolysin having a second enzymatic activity or the domain having the second enzymatic activity, wherein the peptide stretch is selected from the group consisting of synthetic amphipathic peptide, synthetic cationic peptide, synthetic polycationic peptide, synthetic hydrophobic peptide, synthetic antimicrobial peptide (AMP) or naturally occurring AMP; b) incubating the sample for a distinct period, and c) isolating the mycobacterial lysate resulting from step b) thereby obtaining the mycobacterial lysate. Moreover, the present invention relates to the a mycobacterial lysate obtained by the method of the present invention and further to a vaccine composition comprising the mycobacterial lysate, an antibody or antibody fragment generated with the mycobacterial lysate or the vaccine, and a pharmaceutical composition comprising the antibody or antibody fragment.

Mycobacteria are classified as Gram positive bacteria. In comparison to most of the Gram-positive bacteria however, the structure of the cell wall of mycobacteria is different in their composition. The complex structure of the cell wall of mycobacteria consists of a mycolic acid-rich outer membrane which is covalently linked to the arabinogalactan-petidoglycan complex (Hoffmann et al., 2008; Zuber et al., 2008). The mycolic acids are alpha-alkyl, beta-hydroxy C₆₀₋₉₀ fatty acids. The distinct composition of the mycolic acids is dependent on the Mycobacterium species including short saturated alpha, C_{20-25,} and a longer meromycolate chain, the beta-hydroxy branch C₆₀, comprising doublebonds, cyclopropane rings and oxygenated groups. The outer membrane is linked with esterification to the terminal pentaarabinofuranosyl components of arabinogalactan (Payne et al., Molecular Microbiology, 73(3), 2009). The arabinogalactan is covalently linked to peptidoglycan. This covalently linked complex is known as mycolyl-arabinogalactan peptidoglycan (mAGP). This mAGP is known as the cell wall core and builds a stable scaffolding to anchor the outer non-covalently associated lipid and glycoplipids including trehalose 6,6′-dimycolate (TDM or cord factor) (Gil et al., Microbiology, 156, 2010). TDM is a secreted molecule which is important for the pathogenesis of mycobacteria (Brennan, 2003). The cell surface of mycobacteria has the characteristics of a highly hydrophobicity and fastness in view of acids due to the special structure of mAGP and TDM in combination with trehalose 6′-monomycolate. These special properties are leading to the fact that mycobacteria are resistant to dehydration and posses a natural impermeability to nutrients and antibacterial drugs (Gil et al., Microbiology, 156, 2010).

Mycobacterium tuberculosis is the cause for the tuberculosis, an infectious disease which typically affects the lungs. Tuberculosis is a health and life threatening disease. In 2009, 9.4 million new cases of tuberculosis and 1.7 million deaths are counted (Global Tuberculosis Control WHO Report 2010. World Health Organization; Geneva: 2010). Mycobacterium tuberculosis is spread as a primarily respiratory pathogen. Patients with an active infection can transmit the infection by coughing. A major part of infected human patients are not able to eliminate the bacteria completely. This results in the so called “latent” stage, defining a status, wherein the patient is still infected, but does not show any symptoms of the disease. This latent stage however can change in some patients due to a reactivation of the infection resulting in an active stage of tuberculosis. Typically, an infection with mycobacterium tuberculosis starts with the inhalation of the bacteria, followed by the presentation by antigen-presenting immune cells, such as macrophages or dendritic cells, in the airway. Infected macrophages include mycobacteria in intracellular vesicles. However, these vesicles are not accessible for a fusion with lysosomes, which would result in a killing of the mycobacteria. After activation of the infected macrophages with a specific T_{H}1-cell, a lysosomal fusion occurs. Further to this first infection step, infected macrophages recruit uninfected macrophages. Thereby a so called granuloma is formed. The structure of such a granuloma, which is also called caseous granuloma because of the “cheese-like” look, comprises macrophages surrounding a necrotic area with adjacent of B and T cells.

Vaccination against tuberculosis has been conducted with attenuated mycobacterial vaccination strain (BCG) to achieve a protection against tuberculosis. However, this vaccination has been associated with severe side-effects and complications. Furthermore, the BCG vaccination achieved only a reduced effectiveness. Therefore, this vaccination is not recommended any more. The BGC vaccination was not able to reduce the global spread of tuberculosis. Vaccination against mycobacteria is also difficult since immune responses against the mycobacterial vaccination strains can be attenuated due to non-pathogen mycobacteria species living in soil or drinking water. Having been in contact with these non-pathogen mycobacteria before, reduces immune responses against mycobacteria strains which are foreseen for vaccination. This provides further disadvantages to achieve sufficient immune responses in particular in tropical or subtropical regions.

Among the current strategies under investigation, the generation of a new vaccine based on the properties of the complex and highly immunogenic mycobacterial cell wall is a very attractive route, as the complex mycobacterial cell wall is known to affect the immune response. Some of the most immunogenic antigens are located on the cell wall or secreted thereof. Therefore, the mycobacterial cell wall is discussed in the literature as research target to design improved vaccines against tuberculosis (see e.g. Morandi M, Sali M, Manganelli R, Delogu G. J Infect Dev Ctries. 2013 Mar. 14; 7(3):169-81).

The generation of fragments deriving from the mycobacterial cell wall appears to be interesting for use as a vaccine. However, the mycobacterial cell wall is much more complex, and the peptidoglycan layer is covered by a membrane.

Mycobacteria in general can be classified into several major groups for purpose of diagnosis and treatment: M. tuberculosis complex, which can cause tuberculosis: M. tuberculosis, M. bovis, M. africanum, and M. microti; M. leprae, which causes Hansen's disease or leprosy; Nontuberculous mycobacteria (NTM) define all the other mycobacteria, which can cause pulmonary disease resembling tuberculosis, lymphadenitis, skin disease, or disseminated disease. Mycobacteria not only cause human infections but also animal infections as well, e.g. Mycobacterium avium, Mycobacterium avium subsp. paratuberculosis, Mycobacerium bovis.

Mycobacteriophages are a subgroup of bacteriophages, which are bacterial viruses, which target mycobacterial hosts. In view of the special structure and composition of the cell wall of mycobacteria, it is necessary for the mycobacteriophages to degrade the peptidoglycan layer and further to lyse the mycolic acid-rich outer membrane attached to the mAGP complex.

Various types of agents having bactericidal or bacteriostatic activity are known, e.g. antibiotics, endolysins, antimicrobial peptides such as defensins. Increasingly microbial resistance to antibiotics, however, is creating difficulties in treating more and more infections caused by bacteria.

Endolysins are peptidoglycan hydrolases encoded by bacteriophages (or bacterial viruses). They are synthesized during late gene expression in the lytic cycle of phage multiplication and mediate the release of progeny virions from infected cells through degradation of the bacterial peptidoglycan. They are either β(1,4)-glycosylases, transglycosylases, amidases or endopeptidases. Antimicrobial application of endolysins was already suggested in 1991 by Gasson (GB2243611). Although the killing capacity of endolysins has been known for a long time, the use of these enzymes as antibacterials was ignored due to the success and dominance of antibiotics. Only after the appearance of multiple antibiotic resistant bacteria this concept of combating human pathogens with endolysins received interest. A compelling need to develop totally new classes of antibacterial agents emerged and endolysins used as ‘enzybiotics’—a hybrid term of ‘enzymes’ and ‘antibiotics’—seem to meet this need. In 2001, Fischetti and coworkers demonstrated for the first time the therapeutic potential of bacteriophage Cl endolysin towards group A streptococci (Nelson et al., 2001). Since then many publications have established endolysins as an attractive and complementary alternative to control bacterial infections, particularly by Gram positive bacteria. Subsequently different endolysins against other Gram positive pathogens such as Streptococcus pneumoniae (Loeffler et al., 2001), Bacillus anthracis (Schuch et al., 2002), S. agalactiae (Cheng et al., 2005) and Staphylococcus aureus (Rashel et al., 2007) have proven their efficacy as enzybiotics.

Distinct endolysins have been identified in mycobacteriophages (Payne and Hatfull, Plos ONE, 7(3), 2012; Payne et al., Mol Microbiol, 73(3), 2009). These particular endolysins are able to break down the mycobacterial cell wall characterized by the mycol-rich mycobacterial outer membrane attached to an arabinogalactan layer which is in turn linked to the peptidoglycan. These particular phage endolysins can be assigned to two groups, (i) enzymes that cleave the peptidoglycan, and (ii) enzymes that cleave the mycolic acid and arabinogalactan layer.

Antimicrobial peptides (AMPs) represent an important component of the innate immunity against infections against bacteria. Several antimicrobial peptides have been identified which possess an effect against mycobacteria. These antimicrobial peptides are involved not only in the killing of mycobacteria but also in the modulation of the immune defense in form of the secretion of cytokines and chemokines (Shin and Jo, Immune Network, 11(5), 2011).

Antimicrobial peptides (AMPs) represent a wide range of short, cationic or amphipathic, gene encoded peptide antibiotics that can be found in virtually every organism. Different AMPs display different properties, and many peptides in this class are being intensively researched not only as antibiotics, but also as templates for cell penetrating peptides. Despite sharing a few common features (e.g., cationicity, amphipathicity and short size), AMP sequences vary greatly, and at least four structural groups (α-helical, β-sheet, extended and looped) have been proposed to accommodate the diversity of the observed AMP conformations. Likewise, several modes of action as antibiotics have been proposed, and it was shown e.g. that the primary target of many of these peptides is the cell membrane whereas for other peptides the primary target is cytoplasmic invasion and disruption of core metabolic functions. AMPs may become concentrated enough to exhibit cooperative activity despite the absence of specific target binding; for example, by forming a pore in the membrane, as is the case for most AMPs. However, this phenomenon has only been observed in model phospholipid bilayers, and in some cases, AMP concentrations in the membrane that were as high as one peptide molecule per six phospholipid molecules were required for these events to occur. These concentrations are close to, if not at, full membrane saturation. As the minimum inhibitory concentration (MIC) for AMPs are typically in the low micromolar range, scepticism has understandably arisen regarding the relevance of these thresholds and their importance in vivo (Melo et al., Nature reviews, Microbiology, 2009, 245).

Cathelicidins are a family of AMPs which are derived from leukocytes and epithelial cells. Currently, the only identified human cathelicidin is hCAP-18/LL-37 Immunstimulatory effects have been reported for cathelicidins (Shin and Jo, Immune Network, 11(5), 2011).

Defensins are a large family of small, cationic or amphipathic, cysteine- and arginine-rich antimicrobial peptides, found in both vertebrates and invertebrates. Defensins are divided into five groups according to the spacing pattern of cysteines: plant, invertebrate, α-, ρ-, and θ-defensins. The latter three are mostly found in mammals. α-defensins are proteins found in neutrophils and intestinal epithelia. β-defensins are the most widely distributed and are secreted by leukocytes and epithelial cells of many kinds. θ-defensins have been rarely found so far e.g. in leukocytes of rhesus macaques. Defensins are active against bacteria, fungi and many enveloped and nonenveloped viruses. However, the concentrations needed for efficient killing of bacteria are mostly high, i.e. in the μ-molar range. Activity of many peptides may be limited in presence of physiological salt conditions, divalent cations and serum. Depending on the content of hydrophobic amino acid residues defensins also show haemolytic activity.

Hepcidin is a cationic amphipathic bactericidal peptide which is primarily produced in the liver. The expression of Hepcidin is induced during infectious and inflammatory conditions. Crucially, Hepcidin is expressed in macrophages after infection with intracellular pathogens Mycobacterium avium and Mycobacterium tuberculosis. Further, hepcidin causes damage to Mycobacterium tuberculosis and thus exerts immediate antimycobacterial activity (Shin and Jo, Immune Network, 11(5), 2011).

Since there are currently no satisfying vaccination compositions available and in view of the difficulties to treat tuberculosis effectively there is a need for new vaccination compositions. In particular in view of the disadvantages of life and/or attenuated vaccines in regard of safety of the vaccine composition, there is a demand to provide vaccines which fulfill such requirements.

Thus, the technical problem underlying the present invention is the provision of a new vaccine composition as well as methods for the preparation thereof.

This technical problem is solved by the subject-matter defined in the claims.

The term “protein” as used herein refers to a linear polymer of amino acid residues linked by peptide bonds in a specific sequence. The amino-acid residues of a protein may be modified by e.g. covalent attachments of various groups such as carbohydrates and phosphate. Other substances may be more loosely associated with the protein, such as heme or lipid, giving rise to the conjugated proteins which are also comprised by the term “protein” as used herein. The protein may be folded in different ways. The various ways in which the protein fold have been elucidated, are in particular with regard to the presence of alpha helices and beta-pleated sheets. The term “protein” as used herein refers to all four classes of proteins being all-alpha, all-beta, alpha/beta and alpha plus beta. Moreover, the term “protein” refers to a complex, wherein the complex refers to a homomer.

The term “fusion protein” as used herein refers to an expression product resulting from the fusion of different nucleic acid sequences. Such a protein may be produced, e.g., in recombinant DNA expression systems. Moreover, the term “fusion protein” as used herein refers to a fusion of a first amino acid sequence having an enzymatic activity, e.g. an endolysin, with a second and a third amino acid sequence. The second amino acid sequence is preferably a peptide stretch, in particular selected from the group consisting of cationic, polycationic, hydrophobic, amphipathic peptides, and antimicrobial peptides. A third amino acid sequence is a protein transduction domain. Preferably, said second and third amino acid sequence is foreign to and not substantially homologous with any domain of the first amino acid sequence. Moreover, the fusion proteins of the present invention also refer to an expression product resulting from the fusion of at least three nucleic acid sequences.

The term “peptide stretch” as used herein refers to any kind of peptide linked to a protein such as an endolysin. In particular the term “peptide stretch” as used herein refers to a peptide stretch selected from the group consisting of cationic, polycationic, hydrophobic, amphipathic peptides, and antimicrobial peptides (AMP), in particular synthetic amphipathic peptide, synthetic cationic peptide, synthetic polycationic peptide, synthetic hydrophobic peptide, synthetic antimicrobial peptide (AMP) or naturally occurring AMP. In the context of the present invention, AMP are understood as peptides, which provide antimycobacterial activity.

However, a peptide stretch in the meaning of the present invention does not refer to His-tags, preferably His₅-tags, His₆-tags, His₇-tags, His₈-tags, His₉-tags, His₁₀-tags, His₁₁-tags, His₁₂-tags, His₁₆-tags and His₂₀tags, Strep-tags, Avi-tags, Myc-tags, Gst-tags, JS-tags, cystein-tags, FLAG-tags or other tags known in the art, thioredoxin or maltose binding proteins (MBP). The term “tag” in contrast to the term “peptide stretch” as used herein refers to a peptide which can be useful to facilitate expression and/or affinity purification of a polypeptide, to immobilize a polypeptide to a surface or to serve as a marker or a label moiety for detection of a polypeptide e.g. by antibody binding in different ELISA assay formats as long as the function making the tag useful for one of the above listed facilitation is not caused by the positively charge of said peptide. However, the His₆-tag may, depending on the respective pH, also be positively charged, but is used as affinity purification tool as it binds to immobilized divalent cations and is not used as a peptide stretch according to the present invention.

The term “peptide” as used herein refers to short polypeptides consisting of from about 2 to about 100 amino acid residues, more preferably from about 4 to about 50 amino acid residues, more preferably from about 5 to about 30 amino acid residues, wherein the amino group of one amino acid residue is linked to the carboxyl group of another amino acid residue by a peptide bond. A peptide may have a specific function. A peptide can be a naturally occurring peptide or a synthetically designed and produced peptide. The peptide can be, for example, derived or removed from a native protein by enzymatic or chemical cleavage, or can be prepared using conventional peptide synthesis techniques (e.g., solid phase synthesis) or molecular biology techniques (see Sambrook, J. et al., Molecular Cloning: A Laboratory Manual, Cold Spring Harbor Press, Cold Spring Harbor, N.Y. (1989)). Preferred naturally occurring peptides are e.g. antimicrobial peptides and defensins. Preferred synthetically produced peptides are e.g. polycationic, amphipathic or hydrophobic peptides. A peptide in the meaning of the present invention does not refer to His-tags, Strep-tags, thioredoxin or maltose binding proteins (MBP) or the like, which are used to purify or locate proteins.

The term “enzymatic activity” as used herein refers to the effect exerted by one or more enzyme(s) or enzyme like substance(s). An enzymatic activity refers in particular to the effects which are exerted by endolysins. The term “enzymatic activity” refers further in particular to the effect of distinct group of enzyme or enzymatic substances which are having the activity of degrading the cell wall of a Mycobacterium species. A group of these enzymes with this distinct characteristics are named as Lysin A (LysA), of the peptidoglycan-cleavage group, which are known or are proposed to cleave (Payne and Hatfull, Plos ONE, 7(3), 2012; Payne et al., Mol Microbiol, 73(3), 2009):
N-acetyl-b-D-muramidase (lysozyme, lytic transglycosylase);
N-acetyl-b-D-glucosaminidase;
N-acetylmuramoyl-L-alanine amidase;
L-alanoyl-D-glutamate (LD) endopeptidase;
c-D-glutamyl-meso-diaminopimelic acid (DL) peptidase;
D-Ala-m-DAP (DD) endopeptidase; and
m-DAP-m-DAP (LD) endopeptidase.

A further group of enzymes are named as Lysin B (LysB). These enzymes hydrolyze the linkage of the mycolic acids to the peptidoglycan-arabinogalactan complex and comprise at least the following or other lipolytic activities:
Esterase (mycolarabinogalactanesterase)
Cutinase
α/β hydrolase

LysB like proteins are described e.g. in Mycobacteriophage Lysin B is a novel mycolylarabinogalactan esterase Kimberly Payne, Qingan Sun, James Sacchettini, Graham F. Hatfull Mol Microbiol. 2009 August; 73(3): 367-381; Mycobacteriophage Ms6 LysB specifically targets the outer membrane of Mycobacterium smegmatis Filipa Gil, Anna E. Grzegorzewicz, Maria João Catalão, João Vital, Michael R. McNeil, Madalena Pimentel Microbiology. 2010 May; 156(Pt 5): 1497-1504.

A person skilled in the art is able to identify an enzymatic activity as mentioned above with applying a suitable test setting for the distinct enzyme or enzymatic activity.

The term “endolysin” as used herein refers to an enzyme which is a peptidoglycan hydrolase naturally encoded by bacteriophages or bacterial viruses and which is suitable to hydrolyse bacterial cell walls. According to the present invention “endolysins” may derive from mycobacteriophages. Thus, “endolysins” are in particular enzymes such as Lysin A, LysA, or Lysin A like enzymes or Lysin B, LysB, or Lys B like enzymes. “Endolysins” comprise at least one “enzymatically active domain” (EAD) having at least one or more of the following activities: N-acetyl-b-D-muramidase (lysozyme, lytic transglycosylase), N-acetyl-b-D-glucosaminidase, N-acetyl-muramoyl-L-alanine-amidase (amidase) peptidase, L-alanoyl-D-glutamate (LD) endopeptidase, L-alanyl-D-iso-glutaminyl-meso-diaminopimelic acid (D-Ala-m-DAP) (DD) endopeptidase, or m-DAP-m-DAP (LD) endopeptidase. Furthermore, the EAD is having at least one or more of the following activities: lipolytic activity, cutinase, mycolarabinogalactanesterase, or alpha/beta hydrolase. In addition, the endolysins may contain also regions which are enzymatically inactive, and bind to the cell wall of the host bacteria, the so-called CBDs (cell wall binding domains). The endolysin may contain two or more CBDs. Generally, the cell wall binding domain is able to bind different components on the surface of bacteria. Preferably, the cell wall binding domain is a peptidoglycan binding domain and binds to the bacteria's peptidoglycan structure. The different domains of an endolysin can be connected by a domain linker.

The term “domain linker” as used herein refers to an amino acid sequence functioning to connect single protein domains with one another. As a rule domain linkers form no or only few regular secondary structure like α-helices or β-sheets and can occupy different conformations with the respective structural context. Methods to detect domain linker and properties of linker sequences are well known in the art as e.g. described in Bae et al., 2005, Bioinformatics, 21, 2264-2270 or George & Heringa, 2003, Protein Engineering, 15, 871-879.

The term “deletion” as used herein refers to the removal of 1, 2, 3, 4, 5 or more amino acid residues from the respective starting sequence.

The term “insertion” or “addition” as used herein refers to the insertion or addition of 1, 2, 3, 4, 5 or more amino acid residues to the respective starting sequence.

The term “substitution” as used herein refers to the exchange of an amino acid residue located at a certain position for a different one.

The term “cell wall” as used herein refers to all components that form the outer cell enclosure in particular of a Mycobacterium and thus guarantee their integrity. In particular, the term “cell wall” as used herein refers to in particular to the arabinogalactan layer and the mycolic acid layer of Mycobacteria, but also to membranes or additional layers deposited or attached to the mycolic acid layer, such as capsule-like material, outer protein layer or slimes.

The term “EAD” as used herein refers to the enzymatically active domain of an endolysin. The EAD is responsible for hydrolysing bacterial peptidoglycans. It exhibits at least one enzymatic activity of an endolysin. The EAD can also be composed of more than one enzymatically active module. The term “EAD” is used herein synonymously with the term “catalytic domain”.

As used herein, the term “cationic peptide” refers to a synthetic peptide having positively charged amino acid residues. Preferably a cationic peptide has a pKa-value of 9.0 or greater. Typically, at least four of the amino acid residues of the cationic peptide can be positively charged, for example, lysine or arginine. “Positively charged” refers to the side chains of the amino acid residues which have a net positive charge at about physiological conditions. The term “cationic peptide” as used herein refers also to polycationic peptides.

The term “polycationic peptide” as used herein refers to a synthetically produced peptide composed of mostly positively charged amino acid residues, in particular lysine and/or arginine residues. A peptide is composed of mostly positively charged amino acid residues of at least about 20, 30, 40, 50, 60, 70, 75, 80, 85, 90, 95 or about 100% of the amino acid residues are positively charged amino acid residues, in particular lysine and/or arginine residues. The amino acid residues being not positively charged amino acid residues can be neutrally charged amino acid residues and/or negatively charged amino acid residues and/or hydrophobic amino acid residues. Preferably the amino acid residues being not positively charged amino acid residues are neutrally charged amino acid residues, in particular serine and/or glycine.

The term “antimicrobial peptide” (AMP) as used herein refers to any naturally occurring peptide that has microbicidal and/or microbistatic activity in particular against a Mycobacterium species. Thus, the term “antimicrobial peptide” as used herein relates in particular to any peptide having anti-bacterial, anti-infectious, anti-infective and/or germicidal, microbicidal, or bactericidal properties.

The antimicrobial peptide may be a member of the RNAse A super family, a defensin, cathelicidin, granulysin, histatin, psoriasin, dermicidine or hepcidin. The antimicrobial peptide may be naturally occurring in insects, fish, plants, arachnids, vertebrates or mammals. Preferably the antimicrobial peptide may be naturally occurring in insects, fish, plants, arachnids, vertebrates or mammals. Preferably the antimicrobial peptide may be naturally occurring in radish, silk moth, wolf spider, frog, preferably in Xenopus laevis, Rana frogs, more preferably in Rana catesbeiana, toad, preferably Asian toad Bufo bufo gargarizans, fly, preferably in Drosophila, more preferably in Drosophila melanogaster, in Aedes aegypti, in honey bee, bumblebee, preferably in Bombus pascuorum, flesh fly, preferably in Sarcophaga peregrine, scorpion, horseshoe crab, catfish, preferably in Parasilurus asotus, cow, pig, sheep, porcine, bovine, monkey and human.

The term “amphiphatic peptide” as used herein refers to synthetic peptides having both hydrophilic and hydrophobic functional groups. Preferably, the term “amphiphatic peptide” as used herein refers to a peptide having a defined arrangement of hydrophilic and hydrophobic groups e.g. amphipathic peptides may be e.g. alpha helical, having predominantly non polar side chains along one side of the helix and polar residues along the remainder of its surface.

The term “hydrophobic group” as used herein refers to chemical groups such as amino acid side chains which are substantially water insoluble, but soluble in an oil phase, with the solubility in the oil phase being higher than that in water or in an aqueous phase. In water, amino acid residues having a hydrophobic side chain interact with one another to generate a nonaqueous environment. Examples of amino acid residues with hydrophobic side chains are valine, isoleucine, leucine, methionine, phenylalanine, tryptophan, cysteine, alanine, tyrosine, histidine, threonin, serine, proline and glycine residues.

The term “autolysins” refers to enzymes related to endolysins but encoded by bacteria and involved in e.g. cell division. An overview of autolysins is can be found in “Bacterial peptidoglycan (murein) hydrolases. Vollmer W, Joris B, Charlier P, Foster S. FEMS Microbiol Rev. 2008 March; 32(2):259-86”.

The term “bacteriocin” as used herein refers to protein-like, polypeptide-like or peptide-like substances which are able to inhibit the growth of other bacteria. Some bacteriocins are capable of degrading bacterial cell walls like Lysostaphin (degrading Staphylococcus cell walls), Mutanolysin (degrading Streptococcus cell walls) and Enterolysin (degrading Enterococcus cell walls). Preferably said growth inhibition is specifically by means of absorption of said other bacteria to specific receptors of the bacteriocin. A further group of bacteriocins are Nisin-like peptides (Gene encoded antimicrobial peptides, a template for the design of novel anti-mycobacterial drugs. Carroll J, Field D, O'Connor P M, Cotter P D, Coffey A, Hill C, Ross R P, O'Mahony J. Bioeng Bugs. 2010 November-December; 1(6):408-12). In general, bacteriocins are produced by microorganisms. However, the term “bacteriocin” as used herein refers both to an isolated form procuded by a microorganism or to a synthetically produced form, and refers also to variants which substantially retain the activities of their parent bacteriocins, but whose sequences have been altered by insertion or deletion of one or more amino acid residues.

The present invention relates to method for the preparation of a mycobacterial lysate comprising the steps of: a) contacting a sample comprising at least one Mycobacterium species with a composition having the activity of degrading the cell wall of a Mycobacterium species, the composition comprising: (a) a first fusion protein including (i) a first endolysin or a first domain, both having a first enzymatic activity, the enzymatic activity being at least one or more of the following: N-acetyl-b-D-muramidase (lysozyme, lytic transglycosylase), N-acetyl-b-D-glucosaminidase, N-acetylmuramoyl-L-alanine amidase, L-alanoyl-D-glutamate (LD) endopeptidase, c-D-glutamyl-meso-diaminopimelic acid (DL) peptidase, L-alanyl-D-iso-glutaminyl-meso-diaminopimelic acid (D-Ala-m-DAP) (DD) endopeptidase, or m-DAP-m-DAP (LD) endopeptidase; and (ii) at least one peptide stretch fused to the N- or C-terminus of the endolysin having the first enzymatic activity or the domain having the first enzymatic activity, wherein the peptide stretch is selected from the group consisting of synthetic amphipathic peptide, synthetic cationic peptide, synthetic polycationic peptide, synthetic hydrophobic peptide, synthetic antimicrobial peptide (AMP) or naturally occurring AMP; and (b) a second fusion protein including (i) a second endolysin or a second domain, both having a second enzymatic activity, the enzymatic activity being at least one or more of the following: lipolytic activity, cutinase, mycolarabinogalactanesterase, or alpha/beta hydrolase; and (ii) at least one peptide stretch fused to the N- or C-terminus of the endolysin having a second enzymatic activity or the domain having the second enzymatic activity, wherein the peptide stretch is selected from the group consisting of synthetic amphipathic peptide, synthetic cationic peptide, synthetic polycationic peptide, synthetic hydrophobic peptide, synthetic antimicrobial peptide (AMP) or naturally occurring AMP; b) incubating the sample for a distinct period, and c) isolating the mycobacterial lysate resulting from step b) thereby obtaining the mycobacterial lysate.

The composition according to the present invention comprising a first and a second fusion protein allows a degradation of the mycobacterial cell wall in a distinct pattern. This pattern results in a mycobacterial lysate which has advantageous and beneficial properties. The degradation of mycobacteria using the composition of the present invention allows a mild and standardized generation of mycobacterial lysate. This lysate is the result of completely degraded and thus killed mycobacteria. The mycobacterial lysate of the present invention therefore provides fragments, comprising peptide and sugar structures, of the degraded mycobacteria which are characterized to be useful as antigens to provoke a good immune response within a vaccine composition. According to the method of the present invention it is possible to generate mycobacterial fragments in form of so called bacterial ghosts, which represent empty bacterial cells which do not include any longer cytoplasm content. Furthermore, since the mycobacterial lysate does not include any components of living Mycobacteria, the mycobacterial lysate is also safe comparable to a vaccine composition including attenuated Mycobacteria.

The method of the present invention for the preparation of a mycobacterial lysate is a mild method to generate structures for vaccination. In contrast to this, classical ways to prepare a killed vaccine involve for example heat treatment at 50-60° C. or treatment with chemicals like formalin, phenol, mazonin etc. or irradiated by UV. However, these harsh treatments result in a reduced immunogenicity of a killed vaccine, as these standard treatments can denature regions involved in immunogenicity.

Bacterial lysates, which can be regarded as killed vaccines, are only very rarely used as vaccines. The reason for this is that the way of generating the lysate involves methods that can lead to denaturation of regions which are necessary or responsible for immunogenicity.

Therefore, it is surprising and unexpected that the lysate produced with the method of the present invention represents a lysate with good immunogenic characteristics on the one hand side, and which is safe on the other hand side since it does not comprise any living mycobacteria.

In a preferred embodiment of the composition of the present invention the first endolysin is Lysin A (LysA) or the first enzymatic activity of the first domain is exerted by Lysin A (LysA) or Lysin A like enzymes and the second endolysin is Lysin B (LysB) or the second enzymatic activity of the second domain is exerted by Lysin B (LysB) or Lysin B like enzymes.

Examples for the first and second endolysins or the first and second enzymatic activity of the first and second domain are listed in the following table 1.

TABLE 1

| | | type of | | |
|---|---|---|---|---|
| Mycobac- | endolysin/ | endolysin | amino acid | nucleic acid |
| teriophage | domain | or domain | sequence | sequence |
| TM4 | TM4gp29 | Lysin A | SEQ ID NO: 1 | SEQ ID NO: 2 |
| Bxz2 | Bxz2gp11 | Lysin A | SEQ ID NO: 3 | SEQ ID NO: 4 |
| D29 | D29gp10 | Lysin A | SEQ ID NO: 5 | SEQ ID NO: 6 |
| L5 | L5gp10 | Lysin A | SEQ ID NO: 7 | SEQ ID NO: 8 |
| TM4 | TM4gp30 | Lysin B | SEQ ID NO: 9 | SEQ ID |
| | | | | NO: 10 |
| Bxz2 | Bxz2gp12 | Lysin B | SEQ ID | SEQ ID |
| | | | NO: 11 | NO: 12 |
| D29 | D29gp12 | Lysin B | SEQ ID | SEQ ID |
| | | | NO: 13 | NO: 14 |
| L5 | L5gp12 | Lysin B | SEQ ID | SEQ ID |
| | | | NO: 15 | NO: 16 |

In a preferred embodiment the first and the second enzymatic activity of the first and second fusion protein of the composition is exerted by enzymes derived from mycobacteriophages selected from the group consisting of TM4, D29, L5, and Bxz2.

In a further preferred embodiment the peptide stretch of the first and second fusion protein of the composition of the present invention comprises an antimicrobial peptide (AMP), the AMP being selected from the group consisting of Cathelicidins (hCAP-18/LL37), alpha defensins, beta defensins, hepcidin, NK-2, and Ci-MAM-A24.

Examples for antimicrobial peptides according to the present invention are listed in the following table 2.

TABLE 2

| | | nucleic acid |
|---|---|---|
| Peptid | amino acid sequence | sequence |
| LL-37 | LLGDFFRKSKEKIGKEFKRIVQRIKDFLRNLVP | SEQ ID NO: 18 |
| | RTES | |
| | SEQ ID NO: 17 | |
| alpha-defensin | DCYCRIPACIAGERRYGTCIYQGRLWAFCC | SEQ ID NO: 20 |
| | SEQ ID NO: 19 | |
| beta-defensin | NPVSCVRNKGICVPIRCPGSMKQIGTCVGRAV | SEQ ID NO: 22 |
| | KCCRKK | |
| | SEQ ID NO: 21 | |
| Hepcidin | DTHFPICIFCCGCCHRSKCGMCCKT | SEQ ID NO: 24 |
| | SEQ ID NO: 23 | |
| NK-2 | KILRGVCKKIMRTFLRRISKDILTGKK; | SEQ ID NO: 26 |
| | SEQ ID NO: 25 | |
| Ci-MAM-A24 | WRSLGRTLLRLSHALKPLARRSGW | SEQ ID NO: 28 |
| | SEQ ID NO: 27 | |

In a further preferred embodiment the composition of the present invention is having activity of degrading the cell wall of a Mycobacterium species which is selected from the group consisting of Mycobacterium tuberculosis, Mycobacterium microti, Mycobacterium africanum, Mycobacterium bovis, Mycobacterium canettii, Mycobacterium pinnipedii, Mycobacterium caprae, Mycobacterium mungi, Mycobacterium leprae, Mycobacterium ulcerans, Mycobacterium xenopi, Mycobacterium shottsii, Mycobacterium avium, Mycobacterium avium subsp. paratuberculosis, Mycobacterium paratuberculosis, Mycobacterium intracellulare, Mycobacterium smegmatis, Mycobacterium abcessus, Mycobacterium kansasii, Mycobacterium terrae, Mycobacterium nonchromogenicum, Mycobacterium gordonae, and Mycobacterium triviale.

In a preferred embodiment of the present invention the method is conducted, wherein step c) comprises High Performance Liquid chromatography (HPLC), Fast protein liquid chromatography (FPLC), filtration techniques, field flow fractionation, centrifugation or other techniques known as state in the art for the separation of biomolecules from bacterial lysates.

In a preferred embodiment of the method of the present invention, the first fusion protein of the composition exhibits an amino acid sequence selected from the group consisting SEQ ID NO:29, 31, 33, 35, 37, 39, and 41, and wherein the second fusion of the composition protein exhibits an amino acid sequence selected from the group consisting SEQ ID NO:43, 45, 47, 49, 51, 53, and 55.

Specific examples of fusion proteins according to the present invention are listed in the following table.

TABLE 3

| Construct | | amino acid | nucleic acid |
|---|---|---|---|
| number | | sequence | sequence |
| | First fusion protein | | |
| 1 | TM4gp29/LL-37 | SEQ ID NO: 29 | SEQ ID NO: 30 |
| 2 | TM4gp29/LL-37 | SEQ ID NO: 31 | SEQ ID NO: 32 |
| 3 | Bzx2gp11/ | SEQ ID NO: 33 | SEQ ID NO: 34 |
| | alpha-defensin | | |
| 4 | alpha-defensin/ | SEQ ID NO: 35 | SEQ ID NO: 36 |
| | Bzx2gp11 | | |
| 5 | alpha-defensin/ | SEQ ID NO: 37 | SEQ ID NO: 38 |
| | Bzx2gp11/ | | |
| | alpha-defensin | | |
| 6 | beta-defensin/ | SEQ ID NO: 39 | SEQ ID NO: 40 |
| | L5gp10 | | |
| 7 | beta-defensin/ | SEQ ID NO: 41 | SEQ ID NO: 42 |
| | Hepcidin/ | | |
| | L5gp10 | | |
| | Second fusion protein | | |
| 8 | TM4gp30/LL-37 | SEQ ID NO: 43 | SEQ ID NO: 44 |
| 9 | TM4gp30/LL-37 | SEQ ID NO: 45 | SEQ ID NO: 46 |
| 10 | D29gp12/ | SEQ ID NO: 47 | SEQ ID NO: 48 |
| | alpha-defensin | | |
| 11 | alpha-defensin/ | SEQ ID NO: 49 | SEQ ID NO: 50 |
| | D29gp12 | | |
| 12 | alpha-defensin/ | SEQ ID NO: 51 | SEQ ID NO: 52 |
| | D29gp12/ | | |
| | alpha-defensin | | |
| 13 | beta-defensin/ | SEQ ID NO: 53 | SEQ ID NO: 54 |
| | D29gp12 | | |
| 14 | beta-defensin/ | SEQ ID NO: 55 | SEQ ID NO: 56 |
| | Hepcidin/ | | |
| | L5gp12 | | |

In another preferred embodiment of the present invention the enzymes, such as endolysins, autolysins and bacteriocins of the first and second fusion protein according to the present invention comprise modifications and/or alterations of the amino acid sequences. Such alterations and/or modifications may comprise mutations such as deletions, insertions and additions, substitutions or combinations thereof and/or chemical changes of the amino acid residues, e.g. biotinylation, acetylation, pegylation, chemical changes of the amino-, SH- or carboxyl-groups. Said endolysins, autolysins and bacteriocins of the fusion protein according to the present invention exhibit the lytic activity of the respective wild-type endolysin, autolysin and bacteriocin. However, said activity can be the same, higher or lower as the activity of the respective wild-type endolysin, autolysin and bacteriocin. Said activity can be about 10, 20, 30, 40, 50, 60, 70, 80, 90, 100, 110, 120, 130, 140, 150, 160, 170, 180, 190 or about 200% of the activity of the respective wild-type endolysin, autolysin and bacteriocin or even more. The activity can be measured by assays well known in the art by a person skilled in the art as e.g. the plate lysis assay or the liquid lysis assay which are e.g. described in Briers et al., J. Biochem. Biophys Methods 70: 531-533, (2007), Donovan D M, Lardeo M, Foster-Frey J. FEMS Microbiol Lett. 2006 December; 265(1), Payne K M, Hatfull G F PLoS One, 2012.

The peptide stretch and the optional Protein Transduction Domain (PTD) of the fusion proteins according to the present invention may be linked to the endolysin or the domain having an enzymatic activity by additional amino acid residues e.g. due to cloning reasons. Preferably, said additional amino acid residues may be not recognized and/or cleaved by proteases. Preferably the peptide stretch and the PTD may be linked to the endolysin or the domain having an enzymatic activity by at least 1, 2, 3, 4, 5, 6, 7, 8, 9 or 10 additional amino acid residues. In a preferred embodiment the peptide stretch is fused to the N- or C-terminus of the endolysin or the domain having an enzymatic activity by the additional amino acid residues glycine, serine, and alanine (Gly-Ser-Ala). Moreover, the PTD is located on the N-terminus or on the C-Terminus of the first fusion protein or of the second fusion protein according to the invention.

The optional PTD may further comprise additional amino acids on its N- or C-terminus. Preferably the peptide stretch or the PTD comprise the amino acid methionine (Met), or methionine, glycine and serine (Met-Gly-Ser). In another preferred embodiment the first peptide stretch is linked to the N-terminus of the enzyme by the additional amino acid residues, in particular glycine and serine (Gly-Ser) and the second peptide stretch is linked to the N-terminus of the first peptide stretch by the additional amino acid residues, in particular glycine and serine (Gly-Ser). In another preferred embodiment the first peptide stretch is linked to the C-terminus of the enzyme by the additional amino acid residues, in particular glycine and serine (Gly-Ser) and the second peptide stretch is linked to the C-terminus of the first peptide stretch by the additional amino acid residues, in particular glycine and serine (Gly-Ser).

Within the first and second fusion protein according to the present invention the peptide stretch and the PTD are preferably covalently bound to the endolysin or to the domain, both having enzymatic activity. Preferably, the peptide stretch and the PTD consist of at least 5, more preferably at least of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or at least 100 amino acid residues. Especially preferred are peptide stretches and PTDs comprising about 5 to about 100 amino acid residues, about 5 to about 50 or about 5 to about 30 amino acid residues. More preferred are peptide stretches and PTDs comprising about 6 to about 42 amino acid residues, about 6 to about 39 amino acid residues, about 6 to about 38 amino acid residues, about 6 to about 31 amino acid residues, about 6 to about 25 amino acid residues, about 6 to about 24 amino acid residues, about 6 to about 22 amino acid residues, about 6 to about 21 amino acid residues, about 6 to about 20 amino acid residues, about 6 to about 19 amino acid residues, about 6 to about 16 amino acid residues, about 6 to about 14 amino acid residues, about 6 to about 12 amino acid residues, about 6 to about 10 amino acid residues or about 6 to about 9 amino acid residues.

Preferably, the peptide stretches are no tag such as a His-tag, Strep-tag, Avi-tag, Myc-tag, Gst-tag, JS-tag, cystein-tag, FLAG-tag or other tags known in the art and no thioredoxin or maltose binding proteins (MBP). However, the first and second fusion protein according to the present invention may comprise in addition such tag or tags.

More preferably the peptide stretches have the function to lead the first and second fusion protein of the composition of the present invention through the outer membrane but may have activity or may have no or only low activity when administered without being fused to the endolysin or the domain, both having enzymatic activity. The function to lead the first and second fusion protein through the outer membrane of mycobacteria is caused by the potential of the outer membrane or mycolic acid/arabinogalactan or LPS disrupting or permeabilising or destabilizing activity of said peptide stretches in combination with the optional PTDs and the endolysins or the domains. Such outer membrane or LPS disrupting or permeabilising or destabilizing activity of the peptide stretches may be determined in a method as follows: The bacteria cells to be treated are cultured in liquid medium or on agar plates. Then the bacteria cell concentration in the liquid medium is determined photometrically at OD600 nm or the colonies on the agar plates are counted, respectively. Now, the bacteria cells in liquid medium or on the plates are treated with a first and second fusion protein according to the invention. After incubation the bacteria cell concentration in the liquid medium is determined photometrically at OD600 nm or the colonies on the agar plates are counted again. If the first and second fusion protein exhibits such outer membrane or LPS disrupting or permeabilising or destabilizing activity, the bacteria cells are lysed due to the treatment with the fusion protein and thus, the bacteria cell concentration in the liquid medium or the number of the bacteria colonies on the agar plate is reduced. Thus, the reduction in bacteria cell concentration or in the number of bacteria colonies after treatment with the first and second fusion protein is indicative for an outer membrane or LPS disrupting or permeabilising or destabilizing activity of the first and second fusion protein.

Fusion proteins are constructed by linking at least three nucleic acid sequences using standard cloning techniques as described e.g. by Sambrook et al. 2001, Molecular Cloning: A Laboratory Manual. Such a protein may be produced, e.g., in recombinant DNA expression systems. Such fusion proteins according to the present invention can be obtained by fusing the nucleic acids for endolysin and the respective peptide stretches.

A further subject-matter of the present invention relates to an isolated nucleic acid molecule encoding the first fusion protein of the composition of to the present invention or to an isolated nucleic acid molecule encoding the second fusion protein of the composition of the present invention.

Preferably the isolated nucleic acid molecule encoding the first fusion protein of the composition of to the present invention is selected from the group consisting of SEQ ID NO:30, 32, 34, 36, 38, 40, and 42, and wherein the second fusion of the composition protein exhibits an amino acid sequence selected from the group consisting SEQ ID NO:44, 46, 48, 50, 52, 54, and 56.

The present invention further relates to a vector comprising a nucleic acid molecule according to the present invention. Said vector may provide for the constitutive or inducible expression of said fusion protein according to the present invention.

The invention also relates to a method for obtaining said first and second fusion protein of the composition of the present invention from a micro-organism, such as a genetically modified suitable host cell which expresses said fusion proteins. Said host cell may be a microorganism such as bacteria or yeast or an animal cell as e.g. a mammalian cell, in particular a human cell. In one embodiment of the present invention the host cell is a Pichia pastoris cell. The host may be selected due to mere biotechnological reasons, e.g. yield, solubility, costs, etc. but may be also selected from a medical point of view, e.g. a non-pathological bacteria or yeast, human cells.

Another subject-matter of the present invention relates to a method for genetically transforming a suitable host cell in order to obtain the expression of the first and second fusion protein of the composition according to the invention, wherein the host cell is genetically modified by the introduction of a genetic material encoding said fusion proteins into the host cell and obtain their translation and expression by genetic engineering methods well known by a person skilled in the art.

In a preferred embodiment of the present invention, the method for the preparation of a mycobacterial lysate foresees that step b) comprises an incubation temperature preferably of 20° C. to 40° C., and an incubation time preferably of 1 h to 72 h.

A further subject-matter of the invention relates to a mycobacterial lysate according to the present invention which is obtained by a method according to the present invention. The method for the preparation of the mycobacterial lysate foresees using of a composition comprising a first and second fusion protein. The composition allows a good degradation of the mycobacterial cell wall which results in a distinct patter of fragments of the mycobacteria with the mycobacterial lysate. The mycobacterial lysate obtained with the method of the present invention provides fragments, comprising peptide and sugar structures, of the degraded mycobacteria.

A further subject-matter of the invention relates to a vaccine composition for preventing a disease caused by a Mycobacterium species comprising the mycobacterial lysate of the present invention.

According to the present invention, the mycobacterial lysate is the active ingredient within the vaccine composition. The vaccine composition according to the present invention provides the advantages to be safe and to provoke good immune response due to the distinct pattern of fragments present within the mycobacterial lysate.

In a preferred embodiment of the present invention, the vaccine composition is further comprising an adjuvant and/or a pharmaceutical acceptable carrier.

A further subject-matter of the present invention relates to an antibody or an antibody fragment generated by the administration of the mycobacterial lysate according to the present invention or the vaccine composition according to the present invention to a subject.

Different antibody molecules are known in the art. Several types of immunglobulins are IgG, IgM, IgD, IgA or IgE. Further artificially defined antibodies such as bispecific antibodies are further examples.

Several techniques are known to generate antibody fragments (e.g. Morimoto et al., Journal of Biochemical and Biophysical Methods 24, 1992; Brennan at al., Science, 229, 1985). These fragments can also be produced directly by using recombinant cells. Antibody phage libraries are a tool to isolate specific antibody fragments. Fab′-SH fragments can be isolated directly form a E. coli culture and chemically coupled to form F(ab′)₂ fragments (Carter et al., Bio/Technology 10, 1992). Moreover, F(ab′)₂ fragments can be isolated directly from host cell culture. Further techniques for the production of antibody fragments will be apparent to a person skilled in the art. A single chain Fv fragment (scFv) is a further example of an antibody fragment.

The antibodies or antibody fragments according to the present invention possess improved binding abilities. Therefore, these antibodies and antibody fragments are useful tools for research and diagnostic.

Preferably, the antibody of the present invention is a monoclonal or polyclonal antibody.

A further subject-matter of the present invention relates to a pharmaceutical composition comprising the antibody or the antibody fragment according to the present invention for use in preventing or treating an infectious disease caused by a Mycobacterium species.

Further scope of applicability of the present invention will become apparent from the detailed description given hereinafter, however, it should be understood that the detailed description and specific examples, while indicating preferred embodiments of the invention, are given by way of illustration only, since various changes and modifications within the spirit and scope of the invention will become apparent to those skilled in the art from this detailed description. It is to be understood that both the foregoing general description and the following detailed description are exemplary and explanatory only and are not restrictive of the invention, as claimed.

The following examples explain the present invention but are not considered to be limiting. Unless indicated differently, molecular biological standard methods were used, as e.g., described by Sambrock et al., 1989, Molecular Cloning: A Laboratory Manual, 2nd edition, Cold Spring Harbor Laboratory Press, Cold Spring Harbor, N.Y.

### EXAMPLE 1

### Cloning, Expression and Purification of the Respective Fusion Proteins Modified with Various Peptide Stretches at the N-Terminus or the C-Terminus

Proteins

TM4gp29 according to SEQ ID NO: 1 is a Lys A-type endolysin originating from Mycobacteria phage TM4. The endolysin TM4gp29 is encoded by the nucleic acid molecule according to SEQ ID NO: 2. The nucleic acid molecule according to SEQ ID NO: 2 was synthetically produced with a BamH I (5′-GGA TCC-3′) restriction site at the 5′-end of the nucleic acid molecule and an Xho I (5′-CTC GAG-3′) restriction site at the 3′-end of the nucleic acid molecule.

Bxz2gp11 according to SEQ ID NO: 3 is a Lys A-type endolysin originating from Mycobacteria phage Bxz2. The endolysin Bxz2gp11 is encoded by the nucleic acid molecule according to SEQ ID NO: 4. The nucleic acid molecule according to SEQ ID NO: 4 was synthetically produced with a BamH I (5′-GGA TCC-3′) restriction site at the 5′-end of the nucleic acid molecule and an Xho I (5′-CTC GAG-3′) restriction site at the 3′-end of the nucleic acid molecule.

D29gp10 according to SEQ ID NO: 5 is a Lys A-type endolysin originating from Mycobacteria phage D29. The endolysin D29gp10 is encoded by the nucleic acid molecule according to SEQ ID NO: 6. The nucleic acid molecule according to SEQ ID NO: 6 was synthetically produced with a BamH I (5′-GGA TCC-3′) restriction site at the 5′-end of the nucleic acid molecule and an Xho I (5′-CTC GAG-3′) restriction site at the 3′-end of the nucleic acid molecule.

L5gp10 according to SEQ ID NO: 7 is a Lys A-type endolysin originating from Mycobacteria phage L5. The endolysin L5gp10 is encoded by the nucleic acid molecule according to SEQ ID NO: 8. The nucleic acid molecule according to SEQ ID NO: 8 was synthetically produced with a BamH I (5′-GGA TCC-3′) restriction site at the 5′-end of the nucleic acid molecule and an Xho I (5′-CTC GAG-3′) restriction site at the 3′-end of the nucleic acid molecule.

TM4gp30 according to SEQ ID NO: 9 is a Lys B-type endolysin originating from Mycobacteria phage TM4. The endolysin TM4gp30 is encoded by the nucleic acid molecule according to SEQ ID NO: 10. The nucleic acid molecule according to SEQ ID NO: 10 was synthetically produced with a BamH I (5′-GGA TCC-3′) restriction site at the 5′-end of the nucleic acid molecule and an Xho I (5′-CTC GAG-3′) restriction site at the 3′-end of the nucleic acid molecule.

Bxz2gp12 according to SEQ ID NO: 11 is a Lys B-type endolysin originating from Mycobacteria phage Bxz2. The endolysin Bxz2gp12 is encoded by the nucleic acid molecule according to SEQ ID NO: 12. The nucleic acid molecule according to SEQ ID NO: 12 was synthetically produced with a BamH I (5′-GGA TCC-3′) restriction site at the 5′-end of the nucleic acid molecule and an Xho I (5′-CTC GAG-3′) restriction site at the 3′-end of the nucleic acid molecule.

D29gp12 according to SEQ ID NO: 13 is a Lys B-type endolysin originating from Mycobacteria phage D29. The endolysin D29gp12 is encoded by the nucleic acid molecule according to SEQ ID NO: 14. The nucleic acid molecule according to SEQ ID NO: 14 was synthetically produced with a BamH I (5 ‘-GGA TCC-3’) restriction site at the 5 ‘-end of the nucleic acid molecule and an Xho I (5’-CTC GAG-3′) restriction site at the 3′-end of the nucleic acid molecule.

L5gp12 according to SEQ ID NO: 15 is a Lys B-type endolysin originating from Mycobacteria phage L5. The endolysin L5gp12 is encoded by the nucleic acid molecule according to SEQ ID NO: 16. The nucleic acid molecule according to SEQ ID NO: 16 was synthetically produced with a BamH I (5′-GGA TCC-3′) restriction site at the 5′-end of the nucleic acid molecule and an Xho I (5′-CTC GAG-3′) restriction site at the 3′-end of the nucleic acid molecule.

The following peptide stretches in table 1 were used for production of fusion proteins with the endolysins above:

TABLE 4

| Peptide stretch | Protein sequence | Nucleic acid sequence |
|---|---|---|
| LL-37 | SEQ ID NO: 17 | SEQ ID NO: 18 |
| Alpha-defensin | SEQ ID NO: 19 | SEQ ID NO: 20 |
| Beta-defensin | SEQ ID NO: 21 | SEQ ID NO: 22 |
| Hepcidin | SEQ ID NO: 23 | SEQ ID NO: 24 |
| NK-2 | SEQ ID NO: 25 | SEQ ID NO: 26 |
| Ci-MAM-A24 | SEQ ID NO: 27 | SEQ ID NO: 28 |

The nucleic acid molecules encoding the respective peptide stretches were synthetically produced with a Nde I (5′-CAT ATG-3′) restriction site at the 5′-end of the nucleic acid molecule and a BamH I (5′-GGA TCC-3′) restriction site at the 3′-end of the nucleic acid molecule.

Fusion proteins are constructed by linking at least two nucleic acid sequences using standard cloning techniques as described e.g. by Sambrook et al. 2001, Molecular Cloning: A Laboratory Manual. Therefore the nucleic acid molecules encoding the peptide stretches were cleaved in a digest with the respective restriction enzymes Nde I and BamH I and in case of the nucleic acid molecule encoding the peptide stretch for ligation with the proteins the digest was performed with the restriction enzymes Nco I and BamH I. Subsequently the cleaved nucleic acids encoding the peptide stretches were ligated into the pET21 b expression vector (Novagen, Darmstadt, Germany), which was also cleaved in a digest with the respective restriction enzymes Nde I and BamH I before. The cleaved nucleic acid molecule encoding the peptide stretch for ligation with toxic proteins was ligated into a modified pET32 b expression vector (unmodified vector obtainable from Novagen, Darmstadt, Germany), which was also cleaved in a digest with the respective restriction enzymes Nco I and BamH I before. The modification of the pET32b expression vector refers to the deletion of the sequence encoding a S-tag and the central His-tag.

Afterwards, the nucleic acid molecules encoding the proteins were cleaved in a digest with the restriction enzyme BamH I and Xho I, so that the proteins could be ligated into the pET21b expression vector (Novagen, Darmstadt, Germany) and the modified pET32 b expression vector, respectively, which were also cleaved in a digest with the respective restriction enzymes BamH I and Xho I before.

In the case of the peptide stretch, which was introduced by PCR to the C-terminus of the proteins, the resulting fusion protein has a His-tag on the N-terminus, wherein the His-tag is linked to the N-terminus by a linker. For the cloning of the respective nucleic acid molecules the pET32 b expression vector (Novagen, Darmstadt, Germany) was used.

Thus, the nucleic acid molecule encoding the peptide stretch is ligated into the respective vector at the 5′-end of the nucleic acid molecule encoding the respective enzyme. Moreover, the nucleic acid molecule encoding the respective enzyme is ligated into the respective plasmid, so that a nucleic acid molecule encoding a His-tag consisting of six histidine residues is associated at the 3′-end of the nucleic acid molecule encoding the endolysin.

As some fusion proteins may either be toxic upon expression in bacteria, or not homogenous due to protein degradation, the strategy might be to express these fusion proteins fused or linked to other additional proteins. Example for these other additional protein is thioredoxin, which was shown to mediate expression of toxic antimicrobial peptides in E. coli (TrxA mediating fusion expression of antimicrobial peptide CM4 from multiple joined genes in Escherichia coli. Zhou L, Zhao Z, Li B, Cai Y, Zhang S. Protein Expr Purif. 2009 April; 64(2):225-230). In the case of the fusion protein consisting of the N-terminal peptide stretch and the protein, the peptide was ligated into the modified pET32 b expression vector, so that an additional thioredoxin is associated at the 5′-end of the peptide. The thioredoxin could be removed from the expressed fusion protein by the use of enterokinase, therefore between the nucleic acid molecule encoding the peptide and the one encoding the thioredoxin is an enterokinase restriction site introduced.

The sequence of the endolysin-peptide-fusions was controlled via DNA-sequencing and correct clones were transformed into E. coli BL21(DE3) or E. coli BL21(DE3) pLysS (Novagen, Darmstadt, Germany) for protein expression.

Recombinant expression of the fusion proteins according to SEQ ID NO: 29, 31, 35, 37, 41, 43, 45, 47, 49, 51, 53, and 55 is performed in E. coli BL21 (DE3) cells (Novagen, Darmstadt, Germany). The cells were growing until an optical density of OD600 nm of 0.5-0.8 was reached. Then the expression of the fusion protein was induced with 1 mM IPTG (isopropylthiogalactoside) and the expression was performed at 37° C. for a period of 4 hours, alternatively an overnight expression at 16° C. was performed.

E. coli BL21 cells were harvested by centrifugation for 20 mM at 6000 g and disrupted via sonication on ice. Soluble and insoluble fraction of the E. coli crude extract were separated by centrifugation (Sorvall, SS34, 30 mM, 15 000 rpm). All proteins were purified by Ni²⁺ affinity chromatography (Aekta FPLC, GE Healthcare) using the C-terminal 6xHis-tag, encoded by the pET21b or pET32b vectors.

Toxic proteins were expressed using a modified pET32b vector (S-tag and central His-tag deleted), which fuses thioredoxin on the N-terminus of the proteins of interest. The vector also contains an enterokinase cleavage site right before the protein of interest. This site allows the proteolytic cleavage between thioredoxin and the protein of interest, which can purified via the remaining C-terminal His-tag. Expressed fusion proteins were not toxic to the host resulting in high yields of produced protein. For antimicrobial function of the fusion protein it was necessary to remove the thioredoxin by proteolytic cleavage. Therefore the fusion protein was cleaved with 2-4 units/mg recombinant enterokinase (Novagen, Darmstadt, Germany) to remove the thioredoxin following the protocol provided by the manufacturer. After enterokinase cleavage the fusion protein was purified via His-tag purification as described below.

The Ni²⁺ affinity chromatography is performed in 4 subsequent steps, all at room temperature:
1. Equilibration of the Histrap FF 5 ml column (GE Healthcare) with up to 10 column volumes of Washing Buffer (20 mM imidazole, 1 M NaCl and 20 mM Hepes on pH 7.4) at a flow rate of 3-5 ml/min
2. Loading of the total lysate (with wanted fusion protein) on the Histrap FF 5 ml column at a flow rate of 3-5 ml/min
3. Washing of the column with up to 10 column volumes of Washing Buffer to remove unbound sample followed by a second washing step with 10% Elution buffer (500 mM imidazole, 0.5 M NaCl and 20 mM Hepes on pH 7.4) at a flow rate of 3-5 ml/min.
4. Elution of bounded fusion proteins from the column with a linear gradient of 4 column volumes of Elution Buffer (500 mM imidazole, 0.5 M NaCl and 20 mM Hepes on pH 7.4) to 100% at a flow rate of 3-5 ml/min.

Purified stock solutions of fusion proteins in Elution Buffer (20 mM Hepes pH 7.4; 0.5 M NaCl; 500 mM imidazole) were at least 90% pure as determined visually on SDS-PAGE gels (data not shown).

Lysin A like activity was controlled in a Chloroform assay. Escherichia coli BL21 transformed with the respective Lysin A variant were grown at 37° C. in LB broth supplemented with 100 mg/mL ampicillin to an OD600 nm of 0.5 and then induced with a final concentration of 1 mM IPTG. One hour after induction, 2% chloroform was added to the cell suspension and OD600 nm was monitored. Chloroform permeabilizes the inner membrane, thus replacing the holin function, and allows the putative lysin to reach its target in the peptidoglycan layer. The reduction in OD600 nm after addition of chloroform to 10 mL of induced clones was recorded.

Lysin B like activity was controlled by enzymatic assays for lipolytic activity like from those described by Payne et al. 2009. Briefly one milliliter of p-nitrophenyl substrates (50 mM) (Sigma) was incubated with 1 mg of the lysine B variants, or 5 ml of a mock purified sample (derived from pET21 or pET32 containing cells) in buffer (20 mM Tris-HCl pH 8.0, 100 mM NaCl, 0.1% Triton X-100) at room temperature for 30 mM Release of p-nitrophenol was determined by measuring absorbance at 420 nm (A420).

### EXAMPLE 2

### Lysing Activity of Fusion Proteins Modified with Various Peptide Stretches on the N-Terminus or the C-Terminus

Mycobacteria were grown to an OD600 of 1.0. If necessary, clumps were dispersed by passing the bacterial suspension several times through a 25-gauge needle. A volume of 500 ml was added to 3 ml top agar containing 1 mM CaCl₂ and poured onto 7H10 agar plates enriched with 1 mM CaCl₂ and OADC (oleic acid, BSA, dextrose and catalase; Difco). For each modified protein, a serial dilution was prepared in storage buffer. Twenty-microlitre volumes of the original stock and of each dilution were pipetted onto the bacterial lawn, and the spots were allowed to dry completely. Plates were incubated for 4 days for the fast growing mycobacteria, and for up to 6 weeks for the slow growing strains, at the optimal temperature for the individual strain.

TABLE 5
Lytic activity of the fusion proteins

| | | Composition | |
|---|---|---|---|
| | | comprising the | |
| First fusion | Second fusion | first and second | |
| protein | protein | fusion protein | control |
| SEQ ID NO 29: − | SEQ ID NO 43: − | SEQ ID NO 29 + | − |
| | | SEQ ID NO 43: ++ | |
| SEQ ID NO 31: − | SEQ ID NO 45: − | SEQ ID NO 31 + | − |
| | | SEQ ID NO 45: ++ | |
| SEQ ID NO 33: − | SEQ ID NO 47: − | SEQ ID NO 33 + | − |
| | | SEQ ID NO 47: + | |
| SEQ ID NO 35: − | SEQ ID NO: 49: − | SEQ ID NO 35 + | − |
| | | SEQ ID NO 49: + | |
| SEQ ID NO 37: − | SEQ ID NO 51: − | SEQ ID NO 37 + | − |
| | | SEQ ID NO 51: ++ | |
| SEQ ID NO 39: − | SEQ ID NO 53: − | SEQ ID NO 39 + | − |
| | | SEQ ID NO 53: + | |
| SEQ ID NO 41: − | SEQ ID NO 55: − | SEQ ID NO 41 + | − |
| | | SEQ ID NO 55: ++ | |

Abbreviations: − no activity; +: small halo; ++: large halo. “halo” defines the area on the bacterial plate where mycobacteria lysis occurred.

### EXAMPLE 3

### Preparation of Mycobacterial Lysates

Mycobacteria were grown to an OD600 of 1.0. Then a buffered composition of LysA like and LysB like fusion proteins according to SEQ ID NO: 29 and SEQ ID NO: 43, since this composition provided very good mycobacterial lysing activity, was added and the bacteria were incubated for at least 60 minutes at room temperature. If needed the bacterial lysate was purified further.

### EXAMPLE 4

### Microscopy of the Lysated Mycobacteria

Mycobacterial cells from the strains LiCC 5463 and LiCC 5464 were pelleted, washed with reaction puffer (50 mM Hepes, 100 mM NaCl, 10 mM MgCl₂, pH 7.4) and resuspended in reaction buffer to a cell number of about 1×10⁷ cells/ml. The cell suspension was mixed in a 9 (cell solution):1 (protein solution) ratio (90 μl cell solution mixed with 10 μl protein solution) with the solution of first and second fusion protein of the composition of the present invention containing a mixture of construct 3, namely Bxz2gp11/alpha-defensin, and construct 10, namely D29gp12/alpha-defensin. The first and second fusion proteins are each with a concentration of 0.3 mg/ml. Alternatively, constructs 4, namely alpha-defensin/Bzx2gp11, and construct 12, namely alpha-defensin/D29gp12/alpha-defensin, or other combinations of first and second fusion proteins have been used. The samples were incubated at 24° C. for one day and 15 μl samples were taken after 1 h, 2 h, 3 h, 4 h, 5 h, and 15 h incubation (overnight) for microscopic analysis. After 2 h increased cell aggregation of the mycobacterial cells was observed. After 4 h the morphology of the mycobacterial cells started to change: the normal rod structure altered to constricted rod structures (data not shown). After 15 h of incubation mostly clusters of mycobacterial cells were observed and less single cells, whereas the single cells seemed hyaline-like. This hyaline-like structure indicates that the bacterial cytoplasm runs out. After this elimination of the mycobacterial cytoplasm, it is possible to remove the bacterial components by purification. Varying the concentration of the compositions comprising the fusion proteins of the present invention and the incubation time of the compositions with the Mycobacteria, either “empty bacterial cells”, which are also named bacterial ghost, with the cytoplasm leached out, or fragments of the bacterial cell wall have been generated. These fragments have been more purified to provide smaller immunogenic fragments of the cell wall or even carbohydrate or protein structures that have been used for immunization. The fragments of the mycobacterial cell wall and/or the bacterial ghosts (generally also described in the following references: Bioeng Bugs. 2010 September-October; 1(5):326-36. doi: 10.4161/bbug.1.5.12540. The Bacterial Ghost platform system: production and applications. Langemann T, Koller V J, Muhammad A, Kudela P, Mayr U B, Lubitz W.) can be further purified by gelfiltration chromatography, ion exchange chromatography or other chromatographic or filtration techniques. Fractions of this purification have been analyzed regarding their immunogenic potential and suitable fractions have been used for vaccination. To test the immunogenicity of the ghosts or fragments, these structures have been administered intraperitoneally into mice or other suitable animals in comparison to suitable buffer controls. The vaccinated animals were then infected with Mycobacteria, and the protective effect of the ghost cells or respective fragments thereof has been analyzed. Protective structures, which mean distinct structural components of the fragments or bacterial ghosts, have been purified and the structural composition analyzed.

After 15 h a 20 μl sample was platted on Middlebrook 7H9 agar plates and incubated at 37° C. In the control sample plate, containing untreated mycobacteria LiCC 5462 or mycobacteria treated with only one fusion protein, a lawn developed after 2 days. In contrast to this, in sample plates with mycobacteria treated with the first and second fusion proteins of the composition of the present invention even after 3 days of incubation a drastically reduced growth could be observed. Thereby a clearly decreased number of living cells or decreased fitness of the living cells in the treated samples has been recognized (data not shown). By transmission Electron Microscopy (EM) cells with a normal rod like structure have been observed in the untreated control samples as well as in samples only treated with one fusion protein solely. In contrast to this, samples treated with the composition comprising a first fusion protein and a second fusion protein according to the present invention showed cells with drastically changed morphology which was club-shaped. The altered appearance in form of a wider extension and less defined outer structure of the single mycobacteria. This altered, club-shaped morphology provides evidence that integrity of the mycobacteria is lost due to the treatment with the compositions of the present invention. Thus, the compositions of the present invention are able to degrade mycobacterial cell walls. It has been further identified that by increasing the concentration of the protein constructs added, the growth of Mycobacteria could be fully inhibited.

Moreover, it has been recognized that besides of the fully inhibition of the Mycobacteria growth it is also possible to generate fragments of the mycobacterial cell wall. Such mycobacterial fragments as well as so called mycobacterial ghosts have been produced by varying both, concentration of the composition of the first and second fusion protein according to the present invention added to the mycobacteria, and the incubation time.

### EXAMPLE 5

### Characterization of the Mycobacterial Cell Wall Fragments

The mycobacterial cell wall fragments as generated and described above have been further characterized. The mycobacterial cell wall fragments have been plated on agar plates and incubated as described. No growth of mycobacteria has been observed. These results show that the cell wall fragments generated by the composition of first and second fusion protein of the present invention are not able any longer to replicate and to produce living myobacteria.

Moreover, the generation of the mycobacterial cell wall fragments resulted in the release of DNA. The released DNA has been determined using the following PCR protocol:

40 μl of M. smegmatis cells of the strain LiCC 5462 in reaction buffer (50 mM Hepes, 10 mM MgCl₂, pH 7.4), with a total cell number of about 1×10⁷ cells/ml, were mixed with a protein solution (Protein storage buffer: 50 mM Tris, 500 mM NaCl, 500 mM Imidazole, 5% glycerol, pH 8.2), containing only one fusion protein or the first and second fusion proteins of the composition of the present invention, in case of the mixtures with the fusion proteins of the invention a concentration of about 0.8 mg/ml was used. The mixtures were incubated at 24° C. for 4 h or 20 h. Subsequently to the incubation the remaining cells and cell fragments were pelleted by centrifugation at 13000 rpm for 10 mM 10 μl of the supernatant were used as template for the 16s PCR reaction.

The 16s PCR reaction is a normal PCR reaction whereas a special primer pair, in this case 27f and 1492r, has been used which allows the amplification of a part of the gene encoding for the 16S ribosomal subunit. If the cells have released the DNA during the incubation with the fusion proteins of the composition of the present invention, which is a sign of cell lysis or cell leakage, the DNA fragment needed for this PCR reaction is present in the supernatant. Thus, this DNA fragment serves then the template. A product band has shown the presence of released DNA in the supernatant, whereas purified DNA of M. smegmatis has been used as a positive control, to show, that the chosen conditions are sufficient. The storage buffer has been used as a negative control to show that no DNA contaminations were present in the storage buffer.

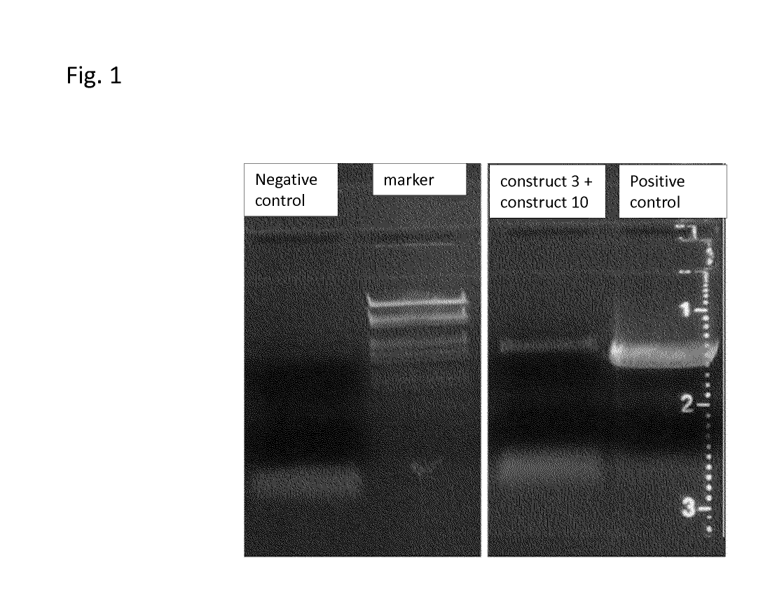

The product bands were detected by Agarose gel electrophoresis. The result is shown in FIG. 1 (exemplarily shown: from left to right-first lane shows negative control, it is clearly visible that no DNA contamination were present—the second lane shows the DNA length standard Peqlab DNA Sizer III (Peqlab/Germany/Erlangen)—the third lane shows the product band produced with the DNA released by incubation for 20 h with construct 3 and construct 10—the fourth lane shows the positive control so the product band produced by using purified DNA of M. smegmatis as template) and cleaned with the Qiagen Gel Extraction Kit (Qiagen/Germany/Hilden) and are sequenced with the primer 27f. The resulting sequences were blasted in NCBI and thereby the origin of the sequence was determined, whereas for the shown bands the origin was M. smegmatis, thereby proofing, that the released DNA was released from the used mycobacterial cells.

PCR Samples:

| | | |
|---|---|---|
| H₂0 bidest. | 30.5 | μl |
| 10x Reaktionspuffer (High Yield) | 5.0 | μl |
| dNTP-Mix (40 mM) | 1.0 | μl |
| Primer 27f (10 pmol/μl) | 1.0 | μl |
| Primer 1492r (10 pmol/μl) | 1.0 | μl |
| Taq-Polymerase (5 u/μl) | 1.0 | μl |

40.0 μl + 10 μl DNA

Template

DNA-Sequences of the Used Primers:

Primer 27f (190):
(SEQ ID NO: 57)
aga gtt tga tcc tgg ctc ag
Primer 1492r (191):
(SEQ ID NO: 58)
tac ggt tac ctt gtt acg act t

PCR Protocol:

| | | |
|---|---|---|
| 95° C. | 2′ | |
| 98° C. | 20″ | |
| 65° C. | 30″ | 15 x (reducing 1° C./cycle) |
| 72° C. | 1′ | |
| 98° C. | 20″ | |
| 50° C. | 30″ | 20 x |
| 72° C. | 1′ | |
| 72° C. | 5′ | |
| 10° C. | ∞ | |

The disruption of Mycobacteria has been sufficient to release the bacterial DNA for detection via PCR. The result according to the agarose gel in FIG. 1 shows that the 16s ribosomal subunit with a size of about 1500 by of the Mycobacterium smegmatis can be detected via PCR. These results show that the treatment with the composition of first and second fusion protein of the present invention allow a complete degradation of the mycobacteria. This degradation of the mycobacteria results in the loss of the integrity of the bacterial cells which is demonstrated by the fact that the DNA of the mycobacteria is accessible and thus detectable. Consequently, the mycobacteria treated with the composition of the present invention are not able to replicate any longer. Therefore, the mycobacterial lysate as produced with the composition of the present invention does not include any living mycobacteria, which means that this lysate fulfills the necessary safety requirements for a vaccine.

1. A method for the preparation of a mycobacterial lysate comprising the steps of:
a) contacting a sample comprising at least one Mycobacterium species with a composition having the activity of degrading the cell wall of a Mycobacterium species, the composition comprising:
(a) a first fusion protein comprising
(i) a first endolysin or a first domain, both having a first enzymatic activity, the enzymatic activity being at least one or more of the following: N-acetyl-b-D-muramidase (lysozyme, lytic transglycosylase), N-acetyl-b-D-glucosaminidase, N-acetylmuramoyl-L-alanine amidase, L-alanoyl-D-glutamate (LD) endopeptidase, c-D-glutamyl-meso-diaminopimelic acid (DL) peptidase, L-alanyl-D-iso-glutaminyl-meso-diaminopimelic acid (D-Ala-m-DAP) (DD) endopeptidase, or m-DAP-m-DAP (LD) endopeptidase; and
(ii) at least one peptide stretch fused to the N- or C-terminus of the endolysin having the first enzymatic activity or the domain having the first enzymatic activity, wherein the peptide stretch is selected from the group consisting of synthetic amphipathic peptide, synthetic cationic peptide, synthetic polycationic peptide, synthetic hydrophobic peptide, synthetic antimicrobial peptide (AMP) or naturally occurring AMP; and
(b) a second fusion protein comprising
(i) a second endolysin or a second domain, both having a second enzymatic activity, the enzymatic activity being at least one or more of the following: lipolytic activity, cutinase, mycolarabinogalactanesterase, or alpha/beta hydrolase; and
(ii) at least one peptide stretch fused to the N- or C-terminus of the endolysin having a second enzymatic activity or the domain having the second enzymatic activity, wherein the peptide stretch is selected from the group consisting of synthetic amphipathic peptide, synthetic cationic peptide, synthetic polycationic peptide, synthetic hydrophobic peptide, synthetic antimicrobial peptide (AMP) or naturally occurring AMP;
b) incubating the sample for a distinct period, and
c) isolating the mycobacterial lysate resulting from step b) thereby obtaining the mycobacterial lysate.

2. The method according to claim 1, wherein the Mycobacterium species is selected from the group consisting of Mycobacterium tuberculosis, Mycobacterium microti, Mycobacterium africanum, Mycobacterium bovis, Mycobacterium canettii, Mycobacterium pinnipedii, Mycobacterium caprae, Mycobacterium mungi, Mycobacterium leprae, Mycobacterium ulcerans, Mycobacterium xenopi, Mycobacterium shottsii, Mycobacterium avium, Mycobacterium avium subsp. paratuberculosis, Mycobacterium paratuberculosis, Mycobacterium intracellulare, Mycobacterium smegmatis, Mycobacterium abcessus, Mycobacterium kansasii, Mycobacterium terse, Mycobacterium nonchromogenicum, Mycobacterium gordonae, and Mycobacterium triviale.

3. The method according to claim 1, wherein step c) comprises High Performance Liquid chromatography (HPLC), Fast protein liquid chromatography (FPLC), filtration techniques, field flow fractionation, centrifugation or other techniques known as state in the art for the separation of biomolecules from bacterial lysates.

4. The method according to claim 1, wherein the first fusion protein of the composition exhibits an amino acid sequence selected from the group consisting SEQ ID NO:29, 31, 33, 35, 37, 39, and 41, and wherein the second fusion of the composition protein exhibits an amino acid sequence selected from the group consisting SEQ ID NO:43, 45, 47, 49, 51, 53, and 55.

5. The method according to claim 1, wherein the step b) comprises an incubation temperature preferably of 20° C. to 40° C., and an incubation time preferably of 1 h to 72 h.

6. A mycobacterial lysate prepared by degrading mycobacteria obtained by a method according to claim 1.

7. A vaccine composition for preventing a disease caused by a Mycobacterium species comprising the mycobacterial lysate according to claim 6.

8. The vaccine composition according to claim 7, further comprising an adjuvant and/or a pharmaceutical acceptable carrier.

9. An antibody or an antibody fragment generated by the administration of the mycobacterial lysate according to claim 6.

10. The antibody according to claim 9, wherein the antibody monoclonal or polyclonal.

11. A method of preventing or treating an infectious disease caused by a Mycobacterium species comprising administering to a subject in need thereof a pharmaceutical composition comprising the antibody or antibody fragment according to claim 9.
